(19) United States
(12) Patent Application Publication
(10) Pub. No.: US 2021/0079334 A1
(43) Pub. Date: Mar. 18, 2021

(54) FERMENTATION SYSTEMS AND METHODS WITH SUBSTANTIALLY UNIFORM VOLUMETRIC UPTAKE RATE OF A REACTIVE GASEOUS COMPONENT

(71) Applicant: Genomatica, Inc., San Diego, CA (US)

(72) Inventors: Jason S. Crater, La Jolla, CA (US); Jefferson Clay Lievense, San Diego, CA (US)

(21) Appl. No.: 16/965,929

(22) PCT Filed: Jan. 28, 2019
(86) PCT No.: PCT/US2019/015583

§ 371 Date: Jul. 29, 2020

Related U.S. Application Data
(60) Provisional application No. 62/623,919, filed on Jan. 30, 2018.

(51) Int. Cl. C12M 1/00; C12M 1/02; C12M 1/36

(52) U.S. Cl. CPC C12M 29/06; C12M 41/48; C12M 27/00

(57) ABSTRACT

Under one aspect, a fermentation system includes a fermentation vessel having a straight wall length L and an inner diameter D. The fermentation system also can include a source of a gas including a reactive gaseous component. The fermentation system also can include spargers spaced apart from one another along the straight wall length L of the fermentation vessel and configured to introduce bubbles of the gas into fermentation broth within the fermentation vessel. The release of the bubbles of the gas by each of the spargers can establish a respective mixing zone within the fermentation broth within the fermentation vessel. Each mixing zone can have substantially the same volumetric uptake rate of the reactive gaseous component by the fermentation broth as each other mixing zone.

### FIELD

This application relates to fermentation systems and methods.

### BACKGROUND

A microbial organism in a fermentation vessel potentially can perform a variety of metabolic processes. At least one of these processes can be limited by availability of a reactive gaseous component within the fermentation broth, such as oxygen in an aerobic metabolic process. In some fermentation vessels, bubbles of a gas including the reactive gaseous component can be introduced into the fermentation broth by a sparger located near the bottom of the vessel. The bubbles of the gas also can mix the fermentation broth within the vessel.

### SUMMARY

Fermentation systems and methods with substantially uniform volumetric uptake rate of a reactive gaseous component are provided herein.

Under one aspect, a fermentation system includes a fermentation vessel having a straight wall length L and an inner diameter D. The fermentation system also can include a source of a gas including a reactive gaseous component. The fermentation system also can include spargers spaced apart from one another along the straight wall length L of the fermentation vessel and configured to introduce bubbles of the gas into fermentation broth within the fermentation vessel. The release of the bubbles of the gas by each of the spargers can establish a respective mixing zone within the fermentation broth within the fermentation vessel. Each mixing zone can have substantially the same volumetric uptake rate of the reactive gaseous component by the fermentation broth as each other mixing zone.

In some configurations, each mixing zone optionally includes an upflow region and a downflow region each established by release of the bubbles of the gas from the respective sparger. In some configurations, in at least one mixing zone, the volumetric uptake rate of the reactive gaseous component optionally is limited by availability of the reactive gaseous component.

In some configurations, the volumetric uptake rate of the reactive gaseous component by the fermentation broth optionally varies by 20% or less across the entire volume of the fermentation broth. In some configurations, the volumetric uptake rate of the reactive gaseous component by the fermentation broth varies by 10% or less across the entire volume of the fermentation broth. In some configurations, the volumetric uptake rate of the reactive gaseous component by the fermentation broth optionally varies by 5% or less across the entire volume of the fermentation broth.

In some configurations, each mixing zone optionally has a volumetric uptake rate of the reactive gaseous component within 20% of that of each other mixing zone. In some configurations, each mixing zone optionally has a volumetric uptake rate of the reactive gaseous component within 10% of that of each other mixing zone. In some configurations, each mixing zone has a volumetric uptake rate of the reactive gaseous component within 5% of that of each other mixing zone.

In some configurations, the fermentation vessel optionally includes a bubble column reactor in which substantially all mixing of the fermentation broth is accomplished by release of the bubbles of the gas by the spargers. Some configurations optionally include three or more spargers. In some configurations, L optionally is equal to or greater than 2D. Optionally, the spargers are spaced apart from one another along the straight wall length L of the fermentation vessel by a distance within 20% of D. Optionally, the spargers are spaced apart from one another along the straight wall length L of the fermentation vessel by a distance within 10% of D. Optionally, the spargers are spaced apart from one another along the straight wall length L of the fermentation vessel by a distance within 5% of D. Optionally, the spargers are spaced apart from one another along the straight wall length L of the fermentation vessel by a distance of D. In some configurations, at least one of the spargers optionally includes a double-ring sparger.

In some configurations, the source includes respective sources of a first gas and a second gas, at least one of the first and second gases including the reactive gaseous component. In some configurations, at least one of the spargers optionally is configured to introduce bubbles including a mixture of the first gas and the second gas into the fermentation broth. In some configurations, at least one of the spargers optionally is configured to introduce bubbles including a different mixture of the first gas and the second gas than does at least one other of the spargers. In some configurations, optionally the first gas is air and the second gas is substantially pure oxygen. In some configurations, optionally the gas is air. In some configurations, optionally the gas is substantially pure oxygen. In some configurations, the reactive gaseous component optionally is selected from the group consisting of oxygen, methane, carbon monoxide, carbon dioxide, nitrogen, and hydrogen. Optionally, the reactive gaseous component is oxygen. Optionally, the reactive gaseous component is carbon dioxide.

Some configurations further include a controller configured to adjust an introduction rate of the reactive gaseous component by at least one of the spargers as a function of time. Optionally, the controller is configured to adjust the introduction rate of the reactive gaseous component by each of the spargers as a function of time. Optionally, responsive to the adjustment of the introduction rate of the reactive gaseous component, a microbial organism in the fermentation broth favors a biological pathway producing a product. In some configurations, the product optionally is selected from the group consisting of 1,4-butanediol, 1,3-butanediol, caprolactam, adipic acid, and 6-amino-caproic acid.

In some configurations, at least one of the spargers optionally has a different introduction rate of the reactive gaseous component than does at least one other of the spargers. In some configurations, optionally each of the spargers includes a ring sparger. In some configurations, optionally at least one of the spargers includes a nozzle or pipe sparger.

In some configurations, responsive to release of the reactive gaseous component within the bubbles of the gas, a microbial organism in the fermentation broth optionally produces a product. Optionally, the product is selected from the group consisting of 1,4-butanediol, 1,3-butanediol, caprolactam, adipic acid, and 6-amino-caproic acid. Optionally, the microbial organism includes a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Optionally, the microbial organism includes a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and Yarrowia lipolytica. Optionally, the microbial organism includes algae or a methanotroph.

Under another aspect, a fermentation method is provided that includes providing a fermentation broth within a fermentation vessel having a straight wall length L and an inner diameter D. The method also can include introducing bubbles of a gas into the fermentation broth by spargers spaced apart from one another along the straight wall length L of the fermentation vessel. The gas can include a reactive gaseous component. The release of the bubbles of the gas by each of the spargers can establish a respective mixing zone within the fermentation broth within the fermentation vessel. Each mixing zone can have substantially the same volumetric uptake rate of the reactive gaseous component by the fermentation broth as each other mixing zone.

In some configurations, in at least one mixing zone, the volumetric uptake rate of the reactive gaseous component is limited by availability of the reactive gaseous component. In some configurations, each mixing zone includes an upflow region and a downflow region each established by release of the bubbles of the gas from the respective sparger. In some configurations, the volumetric uptake rate of the reactive gaseous component by the fermentation broth optionally varies by 20% or less across the entire volume of the fermentation broth. In some configurations, the volumetric uptake rate of the reactive gaseous component by the fermentation broth optionally varies by 10% or less across the entire volume of the fermentation broth. In some configurations, the volumetric uptake rate of the reactive gaseous component by the fermentation broth optionally varies by 5% or less across the entire volume of the fermentation broth.

In some configurations, each mixing zone optionally has a volumetric uptake rate of the reactive gaseous component within 20% of that of each other mixing zone. In some configurations, each mixing zone optionally has a volumetric uptake rate of the reactive gaseous component within 10% of that of each other mixing zone. In some configurations, each mixing zone optionally has a volumetric uptake rate of the reactive gaseous component within 5% of that of each other mixing zone.

In some configurations, the fermentation vessel optionally includes a bubble column reactor in which substantially all mixing of the fermentation broth is accomplished by release of the bubbles of the gas by the spargers. In some configurations, optionally the spargers include three or more spargers. In some configurations, L optionally is equal to or greater than 2D. Optionally, the spargers include a number of spargers equal to L/D rounded up or down to an integer number. In some configurations, the spargers optionally are spaced apart from one another along the straight wall length L of the fermentation vessel by a distance within 20% of D. Optionally, the spargers are spaced apart from one another along the straight wall length L of the fermentation vessel by a distance within 10% of D. Optionally, the spargers are spaced apart from one another along the straight wall length L of the fermentation vessel by a distance within 5% of D. Optionally, the spargers are spaced apart from one another along the straight wall length L of the fermentation vessel by a distance of D. In some configurations, at least one of the spargers optionally includes a double-ring sparger.

In some configurations, introducing the gas includes introducing a first gas and a second gas, at least one of the first and second gases including the reactive gaseous component. Optionally, at least one of the spargers introduces bubbles including a mixture of the first gas and the second gas into the fermentation broth. In some configurations, at least one of the spargers optionally introduces bubbles including a different mixture of the first gas and the second gas than does at least one other of the spargers. In some configurations, optionally the first gas is air and the second gas is substantially pure oxygen.

In some configurations, optionally the gas is air. In some configurations, optionally the gas is substantially pure oxygen. In some configurations, the reactive gaseous component optionally is selected from the group consisting of oxygen, methane, carbon monoxide, carbon dioxide, nitrogen, and hydrogen. Optionally, the reactive gaseous component is oxygen. Optionally, the reactive gaseous component is carbon dioxide.

Some configurations optionally further include adjusting an introduction rate of the reactive gaseous component by at least one of the spargers as a function of time. Some configurations optionally include adjusting the introduction rate of the reactive gaseous component by each of the spargers as a function of time. In some configurations, responsive to the adjustment of the introduction rate of the reactive gaseous component, a microbial organism in the fermentation broth optionally favors a biological pathway producing a product. In some configurations, the product optionally is selected from the group consisting of 1,4-butanediol, 1,3-butanediol, caprolactam, adipic acid, and 6-amino-caproic acid.

In some configurations, at least one of the spargers optionally has a different introduction rate of the reactive gaseous component than does at least one other of the spargers. In some configurations, each of the spargers optionally includes a ring sparger. In some configurations, at least one of the spargers includes a nozzle or pipe sparger.

In some configurations, responsive to release of the reactive gaseous component within the gas, a microbial organism in the fermentation broth optionally produces a product. In some configurations, the product is selected from the group consisting of 1,4-butanediol, 1,3-butanediol, caprolactam, adipic acid, and 6-amino-caproic acid. Optionally, the microbial organism includes a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Optionally, the microbial organism includes a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and Yarrowia lipolytica. Optionally, the microbial organism includes algae or a methanotroph.

### BRIEF DESCRIPTION OF DRAWINGS

FIG. 1 schematically illustrates selected components of a previously known fermentation system.

FIG. 2 schematically illustrates selected components of an exemplary fermentation system, according to some configurations provided herein.

FIGS. 3A-3C schematically illustrate selected components of exemplary fermentation systems, according to some configurations provided herein.

FIG. 4 illustrates a flow of selected operations during an exemplary fermentation method, according to some configurations provided herein.

FIG. 5 is a plot illustrating a simulated exemplary introduction rate of a gas in a fermentation system having a single sparger.

FIG. 6 is a plot illustrating simulated exemplary volumetric uptake rates (VURs) of a reactive gaseous component in different mixing zones of a fermentation system having a single sparger installed at the bottom of the vessel.

FIG. 7 is a plot illustrating oscillations in agitation of varying magnitude to simulate a range of gradients in VUR of a reactive gaseous component in a fermentation system.

FIG. 8 is a plot illustrating a simulated percent oscillation from an average VUR of a reactive gaseous component in a fermentation system having a single sparger installed at the bottom of the vessel.

FIG. 9 is a plot illustrating an exemplary introduction rates of a gas in a fermentation system having a multiple spargers, according to some configurations provided herein.

FIG. 10 is a plot illustrating exemplary VUR of a reactive gaseous component in a fermentation system having multiple spargers, according to some configurations provided herein.

FIG. 11 is a plot illustrating product titer as a function of VUR gradient, according to some configurations provided herein.

FIG. 12 is a plot illustrating product rate as a function of VUR gradient, according to some configurations provided herein.

FIG. 13 is a plot illustrating product yield as a function of VUR gradient, according to some configurations provided herein.

### DETAILED DESCRIPTION

Fermentation systems and methods with substantially uniform volumetric uptake rate of a reactive gaseous component are provided herein.

As noted above, in some previously known fermentation vessels, such as bubble column reactors, bubbles of a gas including a reactive gaseous component can be introduced into the fermentation broth by a sparger located near the bottom of the vessel. In such a system, the volumetric uptake rate (VUR) of the reactive gaseous component by the fermentation broth can vary significantly within the fermentation vessel. Such variance of the VUR can be detrimental to performance of one or more metabolic processes by a microbial organism within the fermentation broth. As provided in greater detail below, configurations of the present fermentation systems and methods can reduce variance of the VUR by the fermentation broth within a fermentation vessel by providing multiple spargers that are spaced apart from one another along the length of the fermentation vessel and that each establishes a respective mixing zone having substantially the same VUR as each other mixing zone, thus enhancing performance of one or more metabolic processes by a microbial organism within the fermentation broth.

### Definitions

As used herein, the term “sparger” is intended to mean an element configured to release bubbles of a gas into a liquid. Spargers include ring spargers, pipe spargers, nozzles, and other types of spargers.

As used herein, the term “bubble” is intended to mean a volume of gas that is at least partially submerged within a volume of liquid. Atoms or molecules within the gas can transfer into the liquid across an interface between the gas and the liquid and also transfer from within the liquid into the gas.

As used herein, the term “reactive gaseous component” is intended to mean an atom or molecule that transfers from a gas into a liquid and that can react with an atom or molecule of the liquid and/or associated with particles and microorganisms in the liquid. For example, the atom or molecule of the gas can transfer from a bubble submerged within the liquid, and then react with an atom or molecule of the liquid. The atom or molecule of the gas can be considered to be a substrate of a reaction and/or a reactant of a reaction. Examples of reactive gaseous components include oxygen, methane, carbon monoxide, carbon dioxide, nitrogen, and hydrogen.

As used herein, the term “react” is intended to mean to be at least partially consumed by a chemical or biological process. For example, a reacting atom or all or part of a reacting molecule can become part of another molecule, or a reacting molecule can be broken down into atoms or smaller molecules. Reactions include, but are not limited to, aerobic reactions in which oxygen is at least partially consumed, and anaerobic reactions in which oxygen substantially is not consumed.

As used herein, the term “aerobic” when used in reference to a culture or growth condition is intended to mean that oxygen is being supplied, whether actively or passively, to the fermentation broth.

As used herein, the term “substantially anaerobic” when used in reference to a culture or growth condition is intended to mean that oxygen is not supplied. Thus the amount of oxygen is less than about 1% of saturation for dissolved oxygen in liquid media when exposed to atmospheric air. The term also is intended to include sealed chambers of liquid or solid medium.

As used herein, the term “mixing zone” is intended to mean a circulation pattern within a liquid under heterogeneous flow conditions. For example, portions of a liquid within one region of a vessel can flow in one direction, and portions of the liquid within another region of the vessel can flow in another direction, such flows establishing a circulation pattern. For example, release of gas bubbles within a bubble column can cause upward flow of liquid within one portion of the column, and downward flow of liquid within another portion of the column, establishing a circulation pattern. For exemplary detail regarding liquid flow and mixing zones in certain types of reactors (including bubble columns), see the following reference, the entire contents of which are incorporated by reference herein: Heijnen et al., “Mass Transfer, Mixing and Heat Transfer Phenomena in Low Viscosity Bubble Column Reactors,” The Chemical Engineering Journal, 28: B21-B42 (1984).

As used herein, the term “bubble column” is intended to mean a vessel that is configured to retain a liquid, and in which substantially all mixing of the liquid is accomplished by release of bubbles of a gas into the liquid. For example, bubble columns exclude impellers, mechanical agitators, or any other element for substantially mixing liquid besides one that releases bubbles of a gas, such as a sparger. A “bubble column reactor” is a bubble column in which one or more reactions is performed.

As used herein, the term “volumetric uptake rate” or “VUR” is intended to mean the rate at which an active fermentation culture consumes a dissolved gaseous component within the fermentation broth. This gaseous component is transferred from a gas bubble across the gas-liquid interface to the liquid fermentation broth where it is then made available to the microorganism.

As used herein, the term “volumetric transfer rate” or “VTR” is intended to mean the rate at which a gaseous component within a bubble transfers to a liquid across the gas-liquid interface. The transfer of a component of a gas into a liquid also can be referred to as “mass transfer.”

As used herein, the term “gas introduction rate” is intended to mean the rate at which a gas is introduced or released into a liquid. The gas can be introduced or released into the liquid in the form of bubbles.

As used herein, “substantially,” “approximately,” “around,” and “about” mean within 20% of the stated value, or within 10% of the stated value, or within 5% of the stated value.

As used herein, the term “non-naturally occurring” when used in reference to a microbial organism or microorganism is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a 1,4-butanediol, 1,3-butanediol, caprolactam, adipic acid, or 6-amino-caproic acid biosynthetic pathway.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Suitable metabolic modifications can be performed on microbial organisms for use in the present fermentation systems and methods.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as algae, methanotrophs, and eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood that such more than one exogenous nucleic acids can be introduced into the host microbial organism on separate nucleic acid molecules, on polycistronic nucleic acid molecules, or a combination thereof, and still be considered as more than one exogenous nucleic acid. For example, a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “bioderived” means derived from or synthesized by a biological organism and can be considered a renewable resource since it can be generated by a biological organism. Such a biological organism, in particular microbial organisms suitable for use in the present fermentation systems and methods, can utilize a variety of carbon sources described herein including feedstock or biomass, such as sugars and carbohydrates obtained from an agricultural, plant, bacterial, or animal source. Alternatively, the biological organism can utilize, for example, atmospheric carbon and/or methanol as a carbon source.

As used herein, the term “biobased” means a product as described herein that is composed, in whole or in part, of a bioderived compound produced by the present fermentation systems and methods. A biobased product is in contrast to a petroleum based product, wherein such a product is derived from or synthesized from petroleum or a petrochemical feedstock.

A “bioderived compound” or a “product,” as used herein, refers to a target molecule or chemical that is derived from or synthesized by a biological organism. In the context of the present fermentation systems and methods, engineered microbial organisms are used to produce a bioderived compound or intermediate thereof. Bioderived compounds (products) that can be produced using the present fermentation systems and methods include, but are not limited to, alcohols, glycols, organic acids, alkenes, dienes, organic amines, organic aldehydes, vitamins, nutraceuticals and pharmaceuticals.

Fermentation Systems

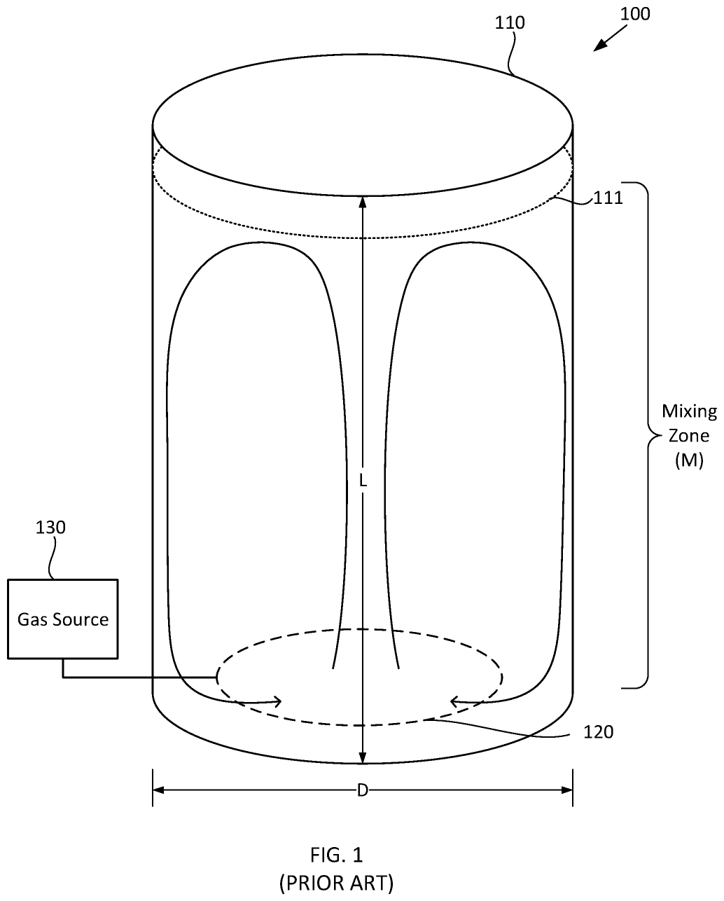

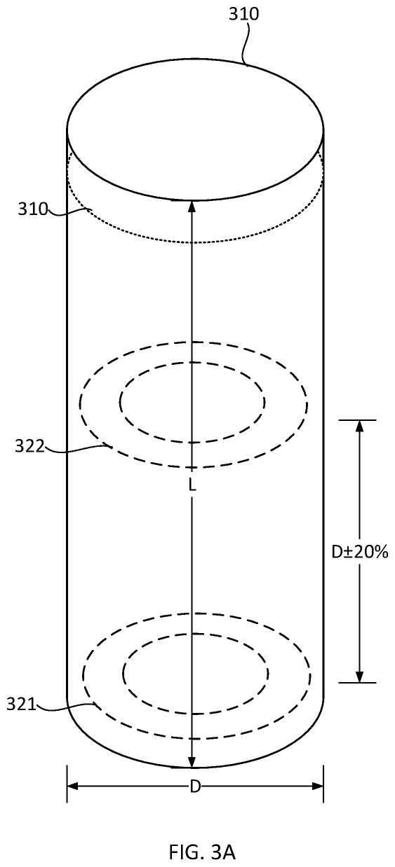

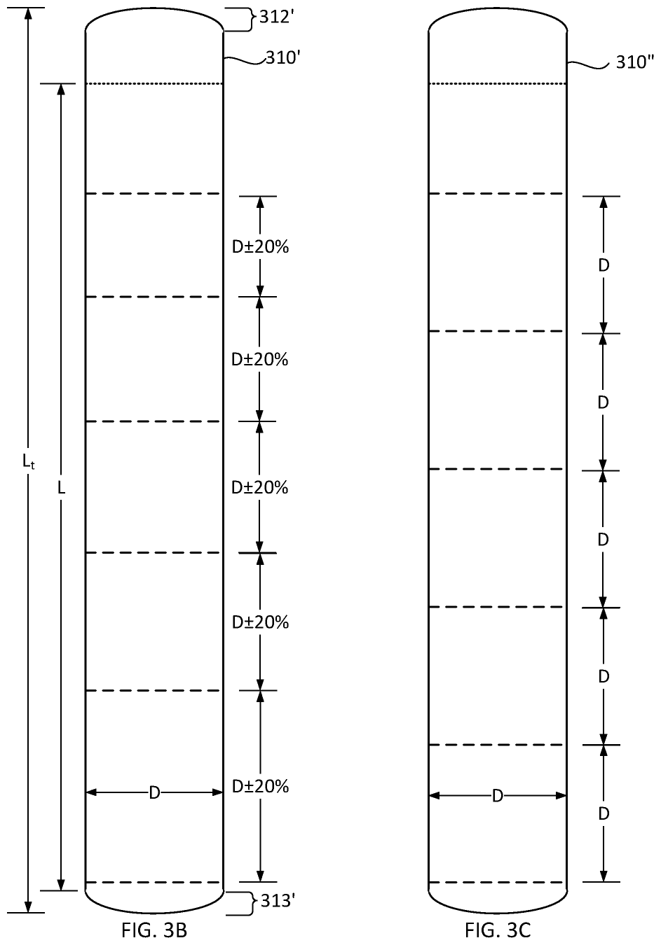

FIG. 1 schematically illustrates selected components of a previously known fermentation system. In FIG. 1 and other figures herein, it should be understood that components are not necessarily drawn to scale. Fermentation system 100 illustrated in FIG. 1 includes fermentation vessel 110, such as a bubble column, having fermentation broth 111 therein. In FIG. 1 and other figures herein, the upper surface of the fermentation broth is indicated by the dotted line. Fermentation vessel 110 can be substantially cylindrical, with a straight wall length L, an inner diameter D, and a circumference. Although not specifically illustrated in FIG. 1, fermentation vessel 110 optionally can be curved on the top and/or bottom in a manner such as illustrated in FIGS. 3B-3C. Fermentation system 100 illustrated in FIG. 1 also includes sparger 120 and gas source 130 that introduce a gas into the fermentation broth that includes a reactive gaseous component. For example, sparger 120 can include a ring sparger that introduces bubbles of the gas from gas source 130 into fermentation broth 111. In FIG. 1 and other figures herein, a ring sparger is indicated by dashed line. The release of the gas bubbles from sparger 120 can establish a mixing zone (M) that extends substantially between the ring sparger and the upper surface of fermentation broth 111. For example, the mixing zone can include an upflow region of fermentation broth 111 that extends substantially between ring sparger 120 and the upper surface of the fermentation broth, and a downflow region of the fermentation broth that extends substantially between the upper surface of the fermentation broth, resulting in circulation and mixing of the fermentation broth such as indicated by the curved arrows.

A microbial organism in fermentation broth 111 illustrated in FIG. 1 can have at least one metabolic process that uses the reactive gaseous component, such as an aerobic metabolic process that uses oxygen. However, the VUR of the reactive gaseous component can vary significantly along the straight wall length L of fermentation vessel 110, e.g., the VUR can be significantly higher near the bottom of vessel 110 and thus near the bottom of mixing zone M than near the upper surface of fermentation broth 111 and thus near the top of mixing zone M. For example, the VUR of the reactive gaseous component from bubbles of the gas into the fermentation broth can be expressed as:

VUR=k_{L}a×(C*−C) (1)

in which k_{L}a is a coefficient that is proportional to the power dissipated by the gas, C* is the concentration of the reactive gaseous component at the gas bubble interface, and C is the concentration of the reactive gaseous component in the bulk fermentation broth. C* is proportional to the product X_{g}P, where X_{g} is the mole fraction of the gaseous reaction component in the gas bubble, and P is the pressure at the bubble exerted by the column of fermentation broth above the bubble. The value of P at the bottom of fermentation vessel 110 can be significantly greater than the value of P at the top of fermentation broth 111 because of the hydrostatic pressure caused by the height of fermentation broth 111 over the bottom of fermentation vessel 110 as compared to the lack of hydrostatic pressure at the upper surface of fermentation broth 111 (at which the height of the fermentation broth is zero and the value of P is based on the pressure of gas over the upper surface of the fermentation broth). In addition, the value of X_{g} at the bottom of fermentation vessel 110 can be significantly greater than the value of X_{g} at the top of fermentation broth 111 because the reactive gaseous component is depleted from the gas as it rises from the bottom to the top of the fermentation vessel. At the same time, there may be other gaseous components which are products of metabolic activity in the liquid which are transferred to the gas phase, further diluting the reactive gaseous component.

In one nonlimiting example, air is the gas that sparger 120 bubbles into the fermentation broth 111, oxygen is the reactive gaseous component, X_{g} is equal to 0.21, P at the bottom of fermentation vessel 110 is equal to 4 atm, P at the top of fermentation broth 111 is equal to 1 atm, half of the oxygen in the incoming air is consumed (reacted), and each mole of consumed oxygen is replaced in the gas phase by a mole of product carbon dioxide. Accordingly, in this example, C* at the bottom of fermentation vessel 110 is equal to 0.21×4, and C* at the top of fermentation broth 111 is equal to 0.105×1. Accordingly, it may be understood that in this particular example, the value of C* at the bottom of fermentation vessel 110 is eight times greater than the value of C* at the top of fermentation broth 111. For other configurations, the value of C* at the bottom of fermentation vessel 110 can be expected to be significantly greater than the value of C* at the top of fermentation broth 111 because of the hydrostatic pressure that fermentation broth 111 causes at the bottom of the vessel, the reactive gaseous component is reduced in the gas phase, and the reactive gaseous component in the gas phase is diluted by other gaseous components which are products of metabolism. As a result, the VUR at the bottom of fermentation vessel 110 can be expected to be significantly greater than at the top of fermentation broth, thus creating a significant gradient in the VUR from the bottom to the top of the fermentation broth. On the other hand, the value of k_{L}a at the bottom of fermentation vessel 110 can be significantly less than the value of k_{L}a at the top of fermentation broth 111 because power is progressively dissipated as the gas bubbles rise and expand with decreasing pressure from bottom to top. In the same nonlimiting example, k_{L}a increases in proportion to the superficial gas velocity raised to the 0.7 power (see Heijnen et al.). The superficial gas velocity is four times greater at the top of the fermentation broth 111 compared to the bottom of the fermentation vessel 110. As a result, the value of k_{L}a at the top of the fermentation broth 111 is 2.64 times greater than the value of k_{L}a at the bottom of the fermentation vessel 110. The net effect of the changes in the values of C* and k_{L}a is that the value of VUR at the bottom of the fermentation vessel 110 is approximately three times greater than the value of k_{L}a at the top of the fermentation broth 111. It also may be understood that as fermentation vessel 110 becomes taller, the difference between the values of C* at the bottom of the fermentation vessel and C* at the top of the fermentation broth can increase, thus increasing the gradient in the VUR between the bottom of the fermentation vessel and the top of the fermentation broth because the difference between the values of C* is only partly offset by the difference in the values of k_{L}a in the calculation of VUR.

Furthermore, the level of fermentation broth 111 within fermentation vessel 110 can change over time. For example, fermentation vessel 110 may be partially full at the beginning of the fermentation process, and then gain volume due to feeding of nutrients during the fermentation, causing the top level of fermentation broth 111 to rise over time. Because changes to the the fermentation broth 111 level can cause changes to the hydrostatic pressure at different levels within fermentation vessel 110, the values of C* at those levels also can be expected to change, only partly offset by the change in the values of k_{L}a in the calculation of VUR. For example, the gradient in the VUR between the bottom of the fermentation vessel 110 and the top of the fermentation broth 111 can change (e.g., increase) as the volume of fermentation broth 111 increases.

Gradients in the VUR of the reactive gaseous component between different regions within fermentation vessel 110 can detrimentally impact a microbial organism's ability to perform certain metabolic process(es). For example, based upon the microbial organism's metabolism being limited by the reactive gaseous component, a gradient in the VUR can be detrimental to performance of the microbial organism because the organism can experience varying levels of reactive gaseous component availability as the organism traverses different areas within fermentation vessel 110. In configurations where the microbial organism is selected to produce a desired product, the production of which product is limited by availability of the reactive gaseous component (such as oxygen), the impact of such varying levels of that component can be severe and can lead to significant reductions in the amount of product produced, e.g., by up to about 20% or even more in one example; the particular performance deviation can be expected to be strain/process dependent. Furthermore, the dynamic supply of the reactive gaseous component can impact the function of one or more metabolic systems (e.g., transcription, translation, and/or regulation), also leading to significant reductions in the amount of product produced.

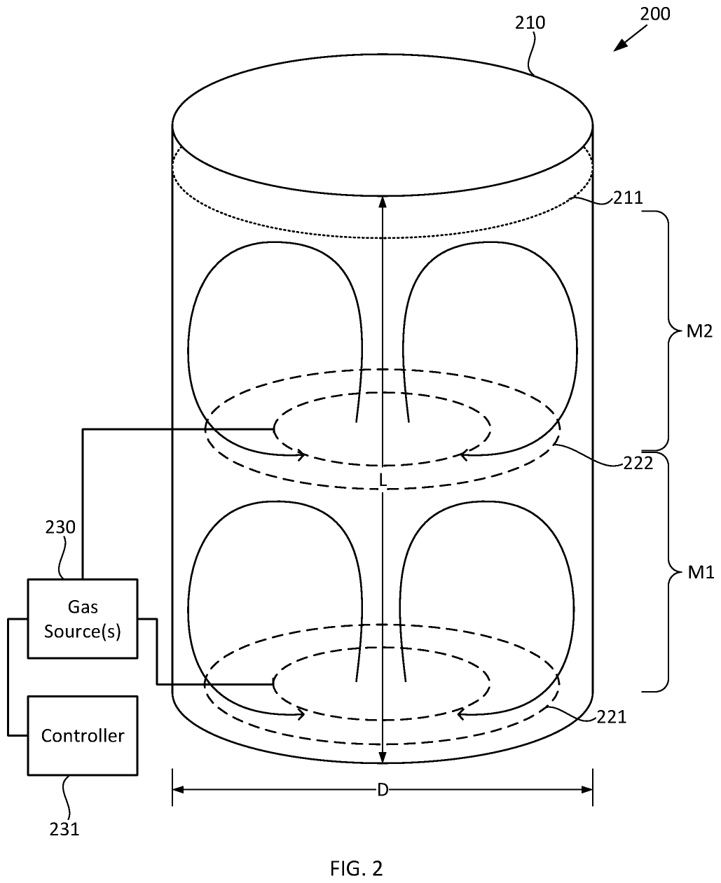

As provided herein, so as to reduce the gradient in the VUR of the reactive gaseous component, a plurality of spargers can be provided within the fermentation vessel that are spaced apart from one another along the length of the fermentation vessel so as to establish a plurality of mixing zones, each of which has substantially the same VUR of the reactive gaseous component as one another. For example, such multiple spargers, each of which optionally can have its own gas flow control system, can allow for the release of additional gas that includes the reactive gaseous component at levels that can increase the values of k_{L}a and/or C* referred to in Equation (1), which can reduce the VUR gradient by maintaining a more even mass transfer distribution of the reactive gaseous component. As described below with reference to FIGS. 2 and 3A-3C, the number of spargers suitably can be determined based on the L/D ratio of the fermentation vessel, and the spacing of the spargers can be determined based on D.

For example, FIG. 2 schematically illustrates selected components of an exemplary fermentation system according to some configurations provided herein. Fermentation system 200 illustrated in FIG. 2 includes fermentation vessel 210 having a fermentation broth 211 therein (the upper surface of which broth is indicated by the dotted line). Optionally, fermentation vessel 211 includes a bubble column reactor in which substantially all mixing of the fermentation broth is accomplished by release of the bubbles of the gas by spargers 221, 222 described in greater detail below. Fermentation vessel 210 can be substantially cylindrical, with a straight wall length L and an inner diameter D. Although not specifically illustrated in FIG. 2, fermentation vessel 210 optionally can be curved on the top and/or bottom in a manner such as illustrated in FIGS. 3B-3C. Fermentation system 200 illustrated in FIG. 2 also includes a source of a gas including a reactive gaseous component, e.g., one or more gas source(s) 230 each of which can be coupled to an optional controller 231 (such as a suitably programmed computer processor) which can be configured so as to control the flow rate of each gas to each sparger 221, 222. Optionally, at least one of spargers 221, 222 has a different introduction rate of the reactive gaseous component than does at least one other of the spargers. For example, sparger 221 can receive a different mixture and/or flow rate of gases from source(s) 231 than does sparger 222, e.g., responsive to suitable control by controller 231.

Fermentation system 200 illustrated in FIG. 2 also includes spargers spaced apart from one another along the straight wall length L of the fermentation vessel and configured to introduce bubbles of the gas into fermentation broth 211 within fermentation vessel 210. For example, in the nonlimiting configuration shown in FIG. 2, the spargers can include first and second spargers 221, 222 (indicated by dashed lines). Optionally, each of the spargers 221, 222 includes or is a ring sparger, which ring sparger optionally can include multiple, attached rings such as illustrated in FIG. 2, or optionally can include a single ring such as illustrated in FIG. 1. As yet another option, one or more of the spargers (and optionally all of the spargers) can include a pipe sparger, nozzle, or other suitable type of sparger. The spargers can be of the same type as one another, or can be of one or more different types than one another. The gas(es) and reactive gaseous component(s) that spargers 221, 221 respectively introduce into the fermentation broth 211 suitably can be selected based on the metabolic needs of the microbial organism within the broth and the desired output of the organism. For example, for aerobic metabolism, the gas can be air. In another example, for aerobic metabolism, the gas can be substantially pure oxygen. Exemplary reactive gaseous components can be selected from the group consisting of oxygen, methane, carbon monoxide, carbon dioxide, nitrogen, and hydrogen, or any other suitable reactive gaseous component. As yet another example, a reactive gaseous component can include a pH adjustant (such as ammonia). Illustratively, providing a pH probe in each mixing zone, and controllably inputting amounts of a pH adjustant through each sparger based on the pH measured by the pH probe, can provide for control, reduction, and/or minimization of pH gradients within and between different mixing zones.

The release of the bubbles of the gas by each of first and second spargers 221, 222 illustrated in FIG. 2 establishes a respective mixing zone M1, M2 within the fermentation broth 211 within the fermentation vessel 210. For example, first mixing zone M1 can extend substantially between first sparger 221 and second sparger 222. For example, first mixing zone M1 can include an upflow region of fermentation broth 211 that extends substantially between first sparger 221 and second sparger 222, and a downflow region of the fermentation broth that extends substantially between second sparger 222 and first sparger 221, resulting in circulation and mixing of the fermentation broth such as indicated by the curved areas in first mixing zone M1. Additionally, second mixing zone M2 can extend substantially between second sparger 222 and the upper surface of fermentation broth 211. For example, second mixing zone M2 can include an upflow region of fermentation broth 211 that extends substantially between second sparger 222 and the upper surface of the fermentation broth, and a downflow region of the fermentation broth that extends substantially between the upper surface of the fermentation broth and second sparger 222, resulting in circulation and mixing of the fermentation broth such as indicated by the curved areas in second mixing zone M2. In some configurations, the upflow region is at and near the horizontal center of fermentation vessel 210, and the downflow region is at and near the horizontal periphery (outer circumference) of the fermentation vessel. Within each mixing zone (e.g., M1 and M2), the upflow region and downflow region each can be established by release of the bubbles of the gas from the respective sparger (e.g., sparger 221 and 222).

In the nonlimiting configuration illustrated in FIG. 2, each mixing zone can have substantially the same VUR of the reactive gaseous component as each other mixing zone. For example, in the nonlimiting configuration illustrated in FIGS. 2, M1 and M2 can have substantially the same VUR as one another. By “substantially the same VUR” it is meant that the difference (or gradient) between the VUR in one mixing zone and the VUR in another mixing zone is sufficiently low that the metabolic processes of a microbial organism in one mixing zone are substantially the same as the metabolic processes of that organism in another mixing zone. For example, each mixing zone (e.g., M1, M2) can have a VUR of the reactive gaseous component within 20% of that of each other mixing zone. In another example, each mixing zone can have a VUR of the reactive gaseous component within 10% of that of each other mixing zone. In another example, each mixing zone can have a VUR of the reactive gaseous component within 5% of that of each other mixing zone. Accordingly, in some configurations, the VUR varies by no more than 20% across the entire volume of the fermentation broth. For example, in some configurations, the VUR varies by no more than 10% across the entire volume of the fermentation broth. For example, in some configurations, the VUR varies by no more than 5% across the entire volume of the fermentation broth.

In some configurations, responsive to release of the reactive gaseous component within the bubbles of the gas, a microbial organism in the fermentation broth can produce a product.

Alcohols that can be produced using the present fermentation systems and methods, including biofuel alcohols, include primary alcohols, secondary alcohols, diols and triols, preferably having C3 to C10 carbon atoms. Alcohols include n-propanol and isopropanol. Biofuel alcohols are preferably C3-C10 and include 1-Propanol, Isopropanol, 1-Butanol, Isobutanol, 1-Pentanol, Isopentenol, 2-Methyl-1-butanol, 3-Methyl-1-butanol, 1-Hexanol, 3-Methyl-1-pentanol, 1-Heptanol, 4-Methyl-1-hexanol, and 5-Methyl-1-hexanol. Diols include propanediols and butanediols, including 1,4 butanediol, 1,3-butanediol and 2,3-butanediol. Fatty alcohols include C4-C27 fatty alcohols, including C12-C18, especially C12-C14, including saturate or unsaturated linear fatty alcohols.

Further exemplary bioderived compounds that can be produced using the present fermentation systems and methods include: (a) 1,4-butanediol and intermediates thereto, such as 4-hydroxybutanoic acid (4-hydroxybutanoate, 4-hydroxybutyrate (4-HB); (b) butadiene (1,3-butadiene) and intermediates thereto, such as 1,4-butanediol, 1,3-butanediol, 2,3-butanediol, crotyl alcohol, 3-buten-2-ol (methyl vinyl carbinol) and 3-buten-1-ol; (c) 1,3-butanediol and intermediates thereto, such as 3-hydroxybutyrate (3-HB), 2,4-pentadienoate, crotyl alcohol or 3-buten-1-ol; (d) adipate, 6-aminocaproic acid (6-ACA), caprolactam, hexamethylenediamine (HMDA) and levulinic acid and their intermediates, e.g. adipyl-CoA, 4-aminobutyryl-CoA; (e) methacrylic acid (2-methyl-2-propenoic acid) and its esters, such as methyl methacrylate and methyl methacrylate (known collectively as methacrylates), 3-hydroxyisobutyrate and/or 2-hydroxyisobutyrate and their intermediates; (f) glycols, including 1,2-propanediol (propylene glycol), 1,3-propanediol, glycerol, ethylene glycol, diethylene glycol, triethylene glycol, dipropylene glycol, tripropylene glycol, neopentyl glycol and bisphenol A and their intermediates; (g) succinic acid and intermediates thereto; and (h) fatty alcohols, which are aliphatic compounds containing one or more hydroxyl groups and a chain of 4 or more carbon atoms, or fatty acids and fatty aldehydes thereof, which are preferably C4-C27 carbon atoms. Fatty alcohols include saturated fatty alcohols, unsaturated fatty alcohols and linear saturated fatty alcohols. Examples fatty alcohols include butyl, pentyl, hexyl, heptyl, octyl, nonyl, decyl, undecyl and dodecyl alcohols, and their corresponding oxidized derivatives, i.e. fatty aldehydes or fatty acids having the same number of carbon atoms. Preferred fatty alcohols, fatty aldehydes and fatty acids have C8 to C18 carbon atoms, especially C12-C18, C12-C14, and C16-C18, including C12, C13, C14, C15, C16, C17, and C18 carbon atoms. Preferred fatty alcohols include linear unsaturated fatty alcohols, such as dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) and unsaturated counterparts including palmitoleyl alcohol (C16 unsaturated; cis-9-hexadecen-1-ol), or their corresponding fatty aldehydes or fatty acids.

1,4-Butanediol and intermediates thereto, such as 4-hydroxybutanoic acid (4-hydroxybutanoate, 4-hydroxybutyrate, 4-FIB), are bioderived compounds that can be made using the present fermentation systems and methods. Suitable bioderived compound pathways and enzymes, methods for screening and methods for isolating are found in: WO2008115840A2 published 25 Sep. 2008 entitled Compositions and Methods for the Biosynthesis of 1,4-Butanediol and Its Precursors; WO2010141780A1 published 9 Dec. 2010 entitled Process of Separating Components of A Fermentation Broth; WO2010141920A2 published 9 Dec. 2010 entitled Microorganisms for the Production of 1,4-Butanediol and Related Methods; WO2010030711A2 published 18 Mar. 2010 entitled Microorganisms for the Production of 1,4-Butanediol; WO2010071697A1 published 24 Jun. 2010 Microorganisms and Methods for Conversion of Syngas and Other Carbon Sources to Useful Products; WO2009094485A1 published 30 Jul. 2009 Methods and Organisms for Utilizing Synthesis Gas or Other Gaseous Carbon Sources and Methanol; WO2009023493A1 published 19 Feb. 2009 entitled Methods and Organisms for the Growth-Coupled Production of 1,4-Butanediol; and WO2008115840A2 published 25 Sep. 2008 entitled Compositions and Methods for the Biosynthesis of 1,4-Butanediol and Its Precursors, which are all incorporated herein by reference.

Butadiene and intermediates thereto, such as 1,4-butanediol, 2,3-butanediol, 1,3-butanediol, crotyl alcohol, 3-buten-2-ol (methyl vinyl carbinol) and 3-buten-1-ol, are bioderived compounds that can be made using the present fermentation systems and methods. In addition to direct fermentation to produce butadiene, 1,3-butanediol, 1,4-butanediol, crotyl alcohol, 3-buten-2-ol (methyl vinyl carbinol) or 3-buten-1-ol can be separated, purified (for any use), and then chemically dehydrated to butadiene by metal-based catalysis. Suitable bioderived compound pathways and enzymes, methods for screening and methods for isolating are found in: WO2011140171A2 published 10 Nov. 2011 entitled Microorganisms and Methods for the Biosynthesis of Butadiene; WO2012018624A2 published 9 Feb. 2012 entitled Microorganisms and Methods for the Biosynthesis of Aromatics, 2,4-Pentadienoate and 1,3-Butadiene; WO2011140171A2 published 10 Nov. 2011 entitled Microorganisms and Methods for the Biosynthesis of Butadiene; WO2013040383A1 published 21 Mar. 2013 entitled Microorganisms and Methods for Producing Alkenes; WO2012177710A1 published 27 Dec. 2012 entitled Microorganisms for Producing Butadiene and Methods Related thereto; WO2012106516A1 published 9 Aug. 2012 entitled Microorganisms and Methods for the Biosynthesis of Butadiene; and WO2013028519A1 published 28 Feb. 2013 entitled Microorganisms and Methods for Producing 2,4-Pentadienoate, Butadiene, Propylene, 1,3-Butanediol and Related Alcohols, which are all incorporated herein by reference.

1,3-Butanediol and intermediates thereto, such as 2,4-pentadienoate, crotyl alcohol or 3-buten-1-ol, are bioderived compounds that can be made using the present fermentation systems and methods. Suitable bioderived compound pathways and enzymes, methods for screening and methods for isolating are found in: WO2011071682A1 published 16 Jun. 2011 entitled Methods and Organisms for Converting Synthesis Gas or Other Gaseous Carbon Sources and Methanol to 1,3-Butanediol; WO2011031897A published 17 Mar. 2011 entitled Microorganisms and Methods for the Co-Production of Isopropanol with Primary Alcohols, Diols and Acids; WO2010127319A2 published 4 Nov. 2010 entitled Organisms for the Production of 1,3-Butanediol; WO2013071226A1 published 16 May 2013 entitled Eukaryotic Organisms and Methods for Increasing the Availability of Cytosolic Acetyl-CoA, and for Producing 1,3-Butanediol; WO2013028519A1 published 28 Feb. 2013 entitled Microorganisms and Methods for Producing 2,4-Pentadienoate, Butadiene, Propylene, 1,3-Butanediol and Related Alcohols; WO2013036764A1 published 14 Mar. 2013 entitled Eukaryotic Organisms and Methods for Producing 1,3-Butanediol; WO2013012975A1 published 24 Jan. 2013 entitled Methods for Increasing Product Yields; and WO2012177619A2 published 27 Dec. 2012 entitled Microorganisms for Producing 1,3-Butanediol and Methods Related Thereto, which are all incorporated herein by reference.

Adipate, 6-aminocaproic acid, caprolactam, hexamethylenediamine and levulinic acid, and their intermediates, e.g. 4-aminobutyryl-CoA, are bioderived compounds that can be made using the present fermentation systems and methods. Suitable bioderived compound pathways and enzymes, methods for screening and methods for isolating are found in: WO2010129936A1 published 11 Nov. 2010 entitled Microorganisms and Methods for the Biosynthesis of Adipate, Hexamethylenediamine and 6-Aminocaproic Acid; WO2013012975A1 published 24 Jan. 2013 entitled Methods for Increasing Product Yields; WO2012177721A1 published 27 Dec. 2012 entitled Microorganisms for Producing 6-Aminocaproic Acid; WO2012099621A1 published 26 Jul. 2012 entitled Methods for Increasing Product Yields; and WO2009151728 published 17 Dec. 2009 entitled Microorganisms for the production of adipic acid and other compounds, which are all incorporated herein by reference.

Methacrylic acid (2-methyl-2-propenoic acid) is used in the preparation of its esters, known collectively as methacrylates (e.g. methyl methacrylate, which is used most notably in the manufacture of polymers). Methacrylate esters such as methyl methacrylate, 3-hydroxyisobutyrate and/or 2-hydroxyisobutyrate and their intermediates are bioderived compounds that can be made using the present fermentation systems and methods. Suitable bioderived compound pathways and enzymes, methods for screening and methods for isolating are found in: WO2012135789A2 published 4 Oct. 2012 entitled Microorganisms for Producing Methacrylic Acid and Methacrylate Esters and Methods Related Thereto; and WO2009135074A2 published 5 Nov. 2009 entitled Microorganisms for the Production of Methacrylic Acid, which are all incorporated herein by reference.

1,2-Propanediol (propylene glycol), n-propanol, 1,3-propanediol and glycerol, and their intermediates are bioderived compounds that can be made using the present fermentation systems and methods. Suitable bioderived compound pathways and enzymes, methods for screening and methods for isolating are found in: WO2009111672A1 published 9 Nov. 2009 entitled Primary Alcohol Producing Organisms; WO2011031897A1 17 Mar. 2011 entitled Microorganisms and Methods for the Co-Production of Isopropanol with Primary Alcohols, Diols and Acids; WO2012177599A2 published 27 Dec. 2012 entitled Microorganisms for Producing N-Propanol 1,3-Propanediol, 1,2-Propanediol or Glycerol and Methods Related Thereto, which are all incorporated herein by referenced.

Succinic acid and intermediates thereto, which are useful to produce products including polymers (e.g. PBS), 1,4-butanediol, tetrahydrofuran, pyrrolidone, solvents, paints, deicers, plastics, fuel additives, fabrics, carpets, pigments, and detergents, are bioderived compounds that can be made using the present fermentation systems and methods. Suitable bioderived compound pathways and enzymes, methods for screening and methods for isolating are found in: EP1937821A2 published 2 Jul. 2008 entitled Methods and Organisms for the Growth-Coupled Production of Succinate, which is incorporated herein by reference.

Primary alcohols and fatty alcohols (also known as long chain alcohols), including fatty acids and fatty aldehydes thereof, and intermediates thereto, are bioderived compounds that can be made using the present fermentation systems and methods. Suitable bioderived compound pathways and enzymes, methods for screening and methods for isolating are found in: WO2009111672 published 11 Sep. 2009 entitled Primary Alcohol Producing Organisms; WO2012177726 published 27 Dec. 2012 entitled Microorganism for Producing Primary Alcohols and Related Compounds and Methods Related Thereto, which are all incorporated herein by reference.

Further suitable bioderived compounds that the microbial organisms can be used to produce using the present fermentation systems and methods can be via acetyl-CoA, including optionally further through acetoacetyl-CoA and/or succinyl-CoA. Exemplary well known bioderived compounds, their pathways and enzymes for production, methods for screening and methods for isolating are found in the following patents and publications: succinate (U.S. publication 2007/0111294, WO 2007/030830, WO 2013/003432), 3-hydroxypropionic acid (3-hydroxypropionate) (U.S. publication 2008/0199926, WO 2008/091627, U.S. publication 2010/0021978), 1,4-butanediol (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,169, WO 2010/141920, U.S. publication 2011/0201068, WO 2011/031897, U.S. Pat. No. 8,377,666, WO 2011/047101, U.S. publication 2011/0217742, WO 2011/066076, U.S. publication 2013/0034884, WO 2012/177943), 4-hydroxybutanoic acid (4-hydroxybutanoate, 4-hydroxybutyrate, 4-hydroxybutryate) (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,155, WO 2010/071697), γ-butyrolactone (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. publication 2011/0217742, WO 2011/066076), 4-hydroxybutyryl-CoA (U.S. publication 2011/0003355, WO 2010/141780, U.S. publication 2013/0034884, WO 2012/177943), 4-hydroxybutanal (U.S. publication 2011/0003355, WO 2010/141780, U.S. publication 2013/0034884, WO 2012/177943), putrescine (U.S. publication 2011/0003355, WO 2010/141780, U.S. publication 2013/0034884, WO 2012/177943), Olefins (such as acrylic acid and acrylate ester) (U.S. Pat. No. 8,026,386, WO 2009/045637), acetyl-CoA (U.S. Pat. No. 8,323,950, WO 2009/094485), methyl tetrahydrofolate (U.S. Pat. No. 8,323,950, WO 2009/094485), ethanol (U.S. Pat. No. 8,129,155, WO 2010/071697), isopropanol (U.S. Pat. No. 8,129,155, WO 2010/071697, U.S. publication 2010/0323418, WO 2010/127303, U.S. publication 2011/0201068, WO 2011/031897), n-butanol (U.S. Pat. No. 8,129,155, WO 2010/071697), isobutanol (U.S. Pat. No. 8,129,155, WO 2010/071697), n-propanol (U.S. publication 2011/0201068, WO 2011/031897), methylacrylic acid (methylacrylate) (U.S. publication 2011/0201068, WO 2011/031897), primary alcohol (U.S. Pat. No. 7,977,084, WO 2009/111672, WO 2012/177726), long chain alcohol (U.S. Pat. No. 7,977,084, WO 2009/111672, WO 2012/177726), adipate (adipic acid) (U.S. Pat. No. 8,062,871, WO 2009/151728, U.S. Pat. No. 8,377,680, WO 2010/129936, WO 2012/177721), 6-aminocaproate (6-aminocaproic acid) (U.S. Pat. No. 8,062,871, WO 2009/151728, U.S. Pat. No. 8,377,680, WO 2010/129936, WO 2012/177721), caprolactam (U.S. Pat. No. 8,062,871, WO 2009/151728, U.S. Pat. No. 8,377,680, WO 2010/129936, WO 2012/177721), hexamethylenediamine (U.S. Pat. No. 8,377,680, WO 2010/129936, WO 2012/177721), levulinic acid (U.S. Pat. No. 8,377,680, WO 2010/129936), 2-hydroxyisobutyric acid (2-hydroxyisobutyrate) (U.S. Pat. No. 8,241,877, WO 2009/135074, U.S. publication 2013/0065279, WO 2012/135789), 3-hydroxyisobutyric acid (3-hydroxyisobutyrate) (U.S. Pat. No. 8,241,877, WO 2009/135074, U.S. publication 2013/0065279, WO 2012/135789), methacrylic acid (methacrylate) (U.S. Pat. No. 8,241,877, WO 2009/135074, U.S. publication 2013/0065279, WO 2012/135789), methacrylate ester (U.S. publication 2013/0065279, WO 2012/135789), fumarate (fumaric acid) (U.S. Pat. No. 8,129,154, WO 2009/155382), malate (malic acid) (U.S. Pat. No. 8,129,154, WO 2009/155382), acrylate (carboxylic acid) (U.S. Pat. No. 8,129,154, WO 2009/155382), methyl ethyl ketone (U.S. publication 2010/0184173, WO 2010/057022, U.S. Pat. No. 8,420,375, WO 2010/144746), 2-butanol (U.S. publication 2010/0184173, WO 2010/057022, U.S. Pat. No. 8,420,375, WO 2010/144746), 1,3-butanediol (U.S. publication 2010/0330635, WO 2010/127319, U.S. publication 2011/0201068, WO 2011/031897, U.S. Pat. No. 8,268,607, WO 2011/071682, U.S. publication 2013/0109064, WO 2013/028519, U.S. publication 2013/0066035, WO 2013/036764), cyclohexanone (U.S. publication 2011/0014668, WO 2010/132845), terephthalate (terephthalic acid) (U.S. publication 2011/0124911, WO 2011/017560, U.S. publication 2011/0207185, WO 2011/094131, U.S. publication 2012/0021478, WO 2012/018624), muconate (muconic acid) (U.S. publication 2011/0124911, WO 2011/017560), aniline (U.S. publication 2011/0097767, WO 2011/050326), p-toluate (p-toluic acid) (U.S. publication 2011/0207185, WO 2011/094131, U.S. publication 2012/0021478, WO 2012/018624), (2-hydroxy-3-methyl-4-oxobutoxy)phosphonate (U.S. publication 2011/0207185, WO 2011/094131, U.S. publication 2012/0021478, WO 2012/018624), ethylene glycol (U.S. publication 2011/0312049, WO 2011/130378, WO 2012/177983), propylene (U.S. publication 2011/0269204, WO 2011/137198, U.S. publication 2012/0329119, U.S. publication 2013/0109064, WO 2013/028519), butadiene (1,3-butadiene) (U.S. publication 2011/0300597, WO 2011/140171, U.S. publication 2012/0021478, WO 2012/018624, U.S. publication 2012/0225466, WO 2012/106516, U.S. publication 2013/0011891, WO 2012/177710, U.S. publication 2013/0109064, WO 2013/028519), toluene (U.S. publication 2012/0021478, WO 2012/018624), benzene (U.S. publication 2012/0021478, WO 2012/018624), (2-hydroxy-4-oxobutoxy)phosphonate (U.S. publication 2012/0021478, WO 2012/018624), benzoate (benzoic acid) (U.S. publication 2012/0021478, WO 2012/018624), styrene (U.S. publication 2012/0021478, WO 2012/018624), 2,4-pentadienoate (U.S. publication 2012/0021478, WO 2012/018624, U.S. publication 2013/0109064, WO 2013/028519), 3-butene-1-ol (U.S. publication 2012/0021478, WO 2012/018624, U.S. publication 2013/0109064, WO 2013/028519), 3-buten-2-ol (U.S. publication 2013/0109064, WO 2013/028519), 1,4-cyclohexanedimethanol (U.S. publication 2012/0156740, WO 2012/082978), crotyl alcohol (U.S. publication 2013/0011891, WO 2012/177710, U.S. publication 2013/0109064, WO 2013/028519), alkene (U.S. publication 2013/0122563, WO 2013/040383, US 2011/0196180), hydroxyacid (WO 2012/109176), ketoacid (WO 2012/109176), wax esters (WO 2007/136762) or caprolactone (U.S. publication 2013/0144029, WO 2013/067432) pathway. The patents and patent application publications listed above that disclose bioderived compound pathways are herein incorporated herein by reference.

In some configurations, the non-naturally occurring microbial organism includes a pathway for production of an alcohol. Accordingly, in some configurations, the alcohol is selected from: (i) a biofuel alcohol, wherein said biofuel is a primary alcohol, a secondary alcohol, a diol or triol including C3 to C10 carbon atoms; (ii) n-propanol or isopropanol; and (iii) a fatty alcohol, wherein said fatty alcohol includes C4 to C27 carbon atoms, C8 to C18 carbon atoms, C12 to C18 carbon atoms, or C12 to C14 carbon atoms. In some aspects, the biofuel alcohol is selected from 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol.

In some configurations, the non-naturally occurring microbial organism includes a pathway for production of an diol. Accordingly, in some embodiments, the diol is a propanediol or a butanediol. In some aspects, the butanediol is 1,4 butanediol, 1,3-butanediol or 2,3-butanediol.

In some embodiments, the non-naturally occurring microbial organism includes a pathway for production of a bioderived compound selected from: (i) 1,4-butanediol or an intermediate thereto, wherein said intermediate is optionally 4-hydroxybutanoic acid (4-HB); (ii) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol; (iii) 1,3-butanediol or an intermediate thereto, wherein said intermediate is optionally 3-hydroxybutyrate (3-HB), 2,4-pentadienoate, crotyl alcohol or 3-buten-1-ol; (iv) adipate, 6-aminocaproic acid, caprolactam, hexamethylenediamine, levulinic acid or an intermediate thereto, wherein said intermediate is optionally adipyl-CoA or 4-aminobutyryl-CoA; (v) methacrylic acid or an ester thereof, 3-hydroxyisobutyrate, 2-hydroxyisobutyrate, or an intermediate thereto, wherein said ester is optionally methyl methacrylate or poly(methyl methacrylate); (vi) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto; (vii) succinic acid or an intermediate thereto; and (viii) a fatty alcohol, a fatty aldehyde or a fatty acid including C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

Accordingly, in some embodiments, the non-naturally occurring microbial organism includes a pathway for production of 1,4-butanediol or an intermediate thereto, wherein said intermediate is optionally 4-hydroxybutanoic acid (4-HB). In some embodiments, the non-naturally occurring microbial organism includes a pathway for production of butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol. In some embodiments, the non-naturally occurring microbial organism includes a pathway for production of 1,3-butanediol or an intermediate thereto, wherein said intermediate is optionally 3-hydroxybutyrate (3-HB), 2,4-pentadienoate, crotyl alcohol or 3-buten-1-ol. In some embodiments, the non-naturally occurring microbial organism includes a pathway for production of adipate, 6-aminocaproic acid, caprolactam, hexamethylenediamine, levulinic acid or an intermediate thereto, wherein said intermediate is optionally adipyl-CoA or 4-aminobutyryl-CoA. In some embodiments, the non-naturally occurring microbial organism includes a pathway for production of methacrylic acid or an ester thereof, 3-hydroxyisobutyrate, 2-hydroxyisobutyrate, or an intermediate thereto, wherein said ester is optionally methyl methacrylate or poly(methyl methacrylate). In some embodiments, the non-naturally occurring microbial organism includes a pathway for production of 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto. In some embodiments, the non-naturally occurring microbial organism includes a pathway for production of succinic acid or an intermediate thereto. In some embodiments, the non-naturally occurring microbial organism includes a pathway for production of a fatty alcohol, a fatty aldehyde or a fatty acid including C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

An exemplary product is 1,4-butanediol. Another exemplary product is 1,3-butanediol. Other exemplary products include one or more of caprolactam, adipic acid, and/or 6-amino-caproic acid.

Microbial organisms that are genetically engineered so as to produce products can include a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Other microbial organisms that are genetically engineered so as to produce products can include a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, and Rhizopus oryzae. and Yarrowia lipolytica. Still other microbial organisms that are genetically engineered so as to produce products can include methanotrophs. Still other microbial organisms that are genetically engineered so as to produce products can include algae.

Further detail is provided below regarding selection of a suitable organism to produce a product, and nutrients that can be included in the fermentation broth so as to cause the microbial organism to produce the product. In some configurations, in at least one mixing zone, the VUR of the reactive gaseous component is limited by availability of the reactive gaseous component. For example, the concentration of the reactive gaseous component in the fermentation broth within that mixing zone can be below saturation. Some organisms, such as Escherichia coli or other organisms such as disclosed elsewhere herein, can be genetically engineered so as to favor one metabolic pathway (such as one that produces a product) over another (such as one that causes the microbial organism to grow) based upon the availability of the reactive gaseous component. The present systems and methods can be used so as to provide a VUR of the reactive gaseous component that is substantially the same in each mixing zone and also provides a value C that causes the microbial organism to favor a metabolic process causing production of the product.

The respective VURs of first and second mixing zones M1, M2 illustrated in FIG. 2 suitably can be obtained based on any suitable combination of the following parameters: the type of spargers used for first and second spargers 221, 222; the spacing of first and second spargers 221, 222 relative to one another and relative to the bottom of fermentation vessel 211, the top of fermentation vessel, and/or the top of fermentation broth 211 (optionally, because the height of the fermentation broth can change over time, the spacing of the spargers can be relative to the expected average top of fermentation broth 211); the size and/or distribution of the gas bubbles respectively released by the first and second spargers 221, 222; the mole fraction of the reactive gaseous component in the gas bubbles respectively released by the first and second spargers 221, 222; the pressure of the gas bubbles respectively released by the first and second spargers 221, 222; and the dimensions of the fermentation vessel 210. It should be appreciated that such parameters suitably can be selected to obtain VURs of respective mixing zones for other fermentation systems including multiple spargers such as provided herein, e.g., such as described herein with reference to FIGS. 3A-3C.

For example, although FIG. 2 illustrates an exemplary configuration including two spargers, e.g., two ring spargers, it should be appreciated that any suitable number, spacing, and type of spargers can be used in any configuration or method provided herein. For example, the present fermentation systems can include three or more spargers, four or more spargers, five or more spargers, six or more spargers, seven or more spargers, eight or more spargers, nine or more spargers, ten or more spargers, fifteen or more spargers, or even twenty or more spargers. All of the spargers can be the same type of sparger as one another, e.g., can all be ring spargers (including but not limited to double-ring spargers such as illustrated in FIG. 2), or at least one of the spargers optionally can be different than at least one other sparger, e.g., at least one sparger can be a ring sparger and/or at least one sparger can be a nozzle or pipe sparger. The greater the L/D ratio of the fermentation vessel, the greater the difference in hydrostatic pressure between the bottom of the vessel and the top of the fermentation broth as discussed above with reference to FIG. 1. By providing a suitable number of spargers that are suitably spaced relative to one another, the respective VURs of mixing zones respectively established by such spargers can be substantially the same as one another. Illustratively, the straight wall length L of the fermentation vessel can be equal to or greater than twice the inner diameter D, and the fermentation system can include a number of spargers equal to L/D rounded up or down to an integer number. For example, for a fermentation vessel having straight wall length L=20 and an inner diameter D=1, the fermentation system can in some configurations include 20 spargers. As another example, for a fermentation vessel having straight wall length L=16 and an inner diameter D=3, the fermentation system can in some configurations include either 5 spargers (L/D rounded down to an integer number) or 6 spargers (L/D rounded up to an integer number). However, it should be appreciated that such numbers of spargers are purely illustrative and not intended to be limiting. Any suitable number of spargers can be provided such that the VURs in different mixing zones are substantially the same as one another, e.g., are within 20% of one another, are within 10% of one another, or are within 5% of one another.

The spargers can be spaced apart from one another by any suitable distance, which distance optionally can be based on the value of D, e.g., can be within 20% of D, within 10% of D, within 5% of D, or exactly D. For example, FIGS. 3A-3C schematically illustrate selected components of exemplary fermentation systems according to some configurations provided herein. In the nonlimiting configuration shown in FIG. 3A, first sparger 321 is spaced apart from second sparger 322 along the straight wall length L of fermentation vessel 310 by a distance within 20% of D, which encompasses values within 10% of D, within 5% of D, and a distance of D. The spacing between second sparger 322 and the top of fermentation broth 311 can in some circumstances be a distance within 20% of D, which encompasses values within 10% of D, within 5% of D, and a distance of D. However, as noted above, the level of the fermentation broth 311 can vary over time. Optionally, the level of the topmost sparger (in FIG. 3A, second sparger 322) is selected such that the sparger is expected to be submerged within the fermentation broth during at least part of the fermentation process. In some configurations, the bottom sparger is positioned sufficiently close to the bottom of the fermentation vessel as to reduce or substantially eliminate the presence of any dead zones (regions lacking sufficient reactive gaseous component for organisms therein to perform reactions). For example, the bottom sparger can be at the base of the straight wall of the fermentation vessel or slightly below that level, e.g., in the bottom dish in configurations including a bottom dish, such as illustrated in FIGS. 3B-3C. For a ring sparger, the bubbles can be released from the underside of the ring, and as such the sparger can be spaced at a suitable distance from the bottom of the vessel to provide room for such bubbles to be released. As another option, an additional smaller sparger can be provided down in the dish so as to provide sufficient mass transfer within the dish.

It should be appreciated that in configurations including more than two spargers, the respective spacings between adjacent spargers can be, but need not necessarily be, the same as one another. For example, the spargers can be spaced unevenly from one another. For example, in the nonlimiting configuration shown in FIG. 3B, a plurality of spargers are spaced apart from one another by a distance within 20% of D, but the distances between adjacent spargers are different from one another, e.g., spargers near the bottom of fermentation vessel 310′ are spaced further apart from one another than are spargers near the top of fermentation vessel 310′. In other configurations (not specifically illustrated), spargers near the bottom of fermentation vessel 310′ can be spaced closer to one another than are spargers near the top of fermentation vessel 310′. In still other configurations, such as shown in FIG. 3C, the distance between adjacent spargers within fermentation vessel 310″ can be the same, e.g., can be equal to D.

Additionally, as noted above with reference to FIGS. 1 and 2, fermentation vessels (such as bubble columns) optionally can be curved on the top and/or bottom. FIGS. 3B-3C illustrate such exemplary curvatures, e.g., in regions 312′ and 313′ in FIG. 3B. As a result of such curvatures, the fermentation vessel can have a total length Lt that is greater than the straight wall length L. As exemplified herein, the number of spargers can be based on the straight wall length L. In alternative configurations, the number of spargers can be based on the total length Lt.

The spargers in the present fermentation systems and methods optionally can release different gases and/or different amounts of the reactive gaseous component into the fermentation broth, respectively. For example, referring again to FIG. 2, gas source(s) 230 can include respective sources of a first gas and a second gas. At least one of the first and second gases (and optionally both) can include the reactive gaseous component. At least one of the spargers can be configured to introduce bubbles including a mixture of the first gas and the second gas into the fermentation broth. For example, in the nonlimiting configuration illustrated in FIG. 2, one or both of spargers 221, 222 can be connected to the sources of the first gas and the second gases so as to receive both gases and generate bubbles of a mixture of both gases. Additionally, or alternatively, at least one of the spargers can be configured to introduce bubbles including a different mixture of the first gas and the second gas than does at least one other of the spargers. For example, in the nonlimiting configuration illustrated in FIG. 2, one or both of spargers 221, 222 can be connected to the sources of the first gas and the second gases so as to receive both gases and generate bubbles of a mixture of both gases, wherein the bubbles from sparger 221 can have a different gas mixture than the bubbles from sparger 222. Additionally, or alternatively, the first gas can be air and the second gas can be substantially pure oxygen. Optionally, the reactive gaseous component is oxygen. As another option, the reactive gaseous component is carbon dioxide. Controller 231 illustrated in FIG. 2 optionally can be configured so as to control which gas(es) are received by which sparger(s), e.g., by opening or closing valves associated with each respective gas source 230.

As another option, controller 231 can be configured to adjust an introduction rate of the reactive gaseous component by at least one of the spargers as a function of time, or to adjust the introduction rate of the reactive gaseous component by each of the spargers as a function of time. For example, responsive to the adjustment of the introduction rate of the reactive gaseous component, a microbial organism in the fermentation broth can favor a biological pathway producing a product, such as 1,4-butanediol, 1,3-butanediol, caprolactam, adipic acid, or 6-amino-caproic acid.

Fermentation Methods

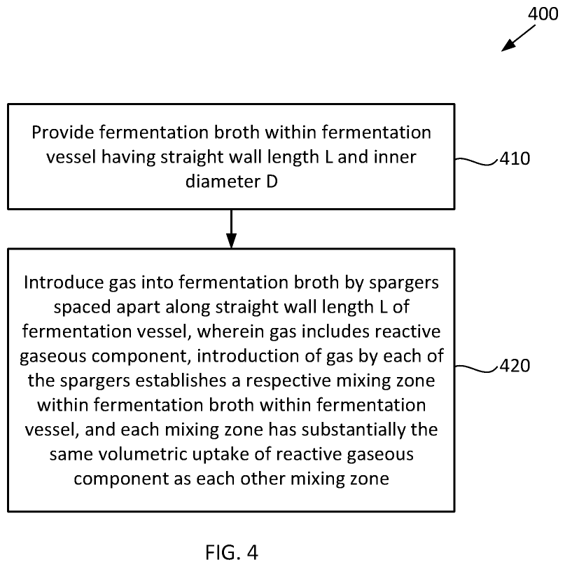

It should be appreciated that the present systems, such as discussed herein with reference to FIGS. 2 and 3A-3B, suitably can be used in any fermentation method. It should also be appreciated that the present fermentation methods can be, but need not necessarily be, used with systems such as illustrated in FIGS. 2 and 3A-3B. For example, FIG. 4 illustrates a flow of selected operations during an exemplary fermentation method according to some configurations provided herein. Fermentation method 400 illustrated in FIG. 4 includes operation 410 including providing a fermentation broth within a fermentation vessel having a straight wall length L and an inner diameter D. For example, fermentation broth can be provided within fermentation vessel 210 described herein with reference to FIG. 2, within fermentation vessel 310 described herein with reference to FIG. 3A, fermentation vessel 310′ described herein with reference to FIG. 3B, fermentation vessel 310″ described herein with reference to FIG. 3C, or any other suitable fermentation vessel. The fermentation broth can include a microbial organism and nutritive components such as described in greater detail below.

Referring gain to FIG. 4, fermentation method 400 can include operation 420 including introducing bubbles of a gas into the fermentation broth by spargers spaced apart from one another along the straight wall length L of the fermentation vessel. For example, in a manner such as described herein with reference to FIG. 2, the gas can include a reactive gaseous component, the release of the bubbles of the gas by each of the spargers (e.g., spargers 221 and 222) can establish a respective mixing zone (e.g., M1 and M2) within the fermentation broth within the fermentation vessel (e.g., vessel 210), and each mixing zone can have substantially the same volumetric uptake rate of the reactive gaseous component as each other mixing zone.

In some configurations of fermentation method 400, in at least one mixing zone, the volumetric uptake rate of the reactive gaseous component is limited by availability of the reactive gaseous component in a manner such as described herein with reference to FIG. 2. Additionally, or alternatively, each mixing zone can include an upflow region and a downflow region each established by release of the bubbles of the gas from the respective sparger in a manner such as described herein with reference to FIG. 2. Additionally, or alternatively, each mixing zone can have a volumetric uptake rate of the reactive gaseous component within 20% of that of each other mixing zone, or within 10% of that of each other mixing zone, or within 5% of that of each other mixing zone, in a manner such as described herein with reference to FIG. 2.

In some configurations of fermentation method 400, the fermentation vessel includes a bubble column reactor in which substantially all mixing of the fermentation broth is accomplished by release of the bubbles of the gas by the spargers, e.g., as described herein with reference to FIG. 2. In some configurations of fermentation method 400, each mixing zone includes an upflow region and a downflow region each established by release of the bubbles of the gas from the respective sparger, e.g., in a manner such as described herein with reference to FIG. 2. In some configurations of fermentation method 400, each mixing zone has a volumetric uptake rate of the reactive gaseous component within 20% of that of each other mixing zone, e.g., in a manner such as described herein with reference to FIG. 2. In some configurations of fermentation method 400, each mixing zone has a volumetric uptake rate of the reactive gaseous component within 10% of that of each other mixing zone, e.g., in a manner such as described herein with reference to FIG. 2. In some configurations of fermentation method 400, each mixing zone has a volumetric uptake rate of the reactive gaseous component within 5% of that of each other mixing zone, e.g., in a manner such as described herein with reference to FIG. 2.

In some configurations of fermentation method 400, the fermentation vessel includes a bubble column reactor in which substantially all mixing of the fermentation broth is accomplished by release of the bubbles of the gas by the spargers, e.g., in a manner such as described herein with reference to FIG. 2. In some configurations of fermentation method 400, the spargers include three or more spargers, e.g., in a manner such as described herein with reference to FIGS. 2 and 3A-3C.

In some configurations of fermentation method 400, L is equal to or greater than 2D, e.g., in a manner such as described herein with reference to FIGS. 2 and 3A-3C. Optionally, In some configurations of fermentation method 400, the spargers include a number of spargers equal to L/D rounded up or down to an integer number, e.g., in a manner such as described herein with reference to FIGS. 2 and 3A-3C. As a further option, the spargers can be spaced apart from one another along the straight wall length L of the fermentation vessel by a distance within 20% of D, e.g., in a manner such as described herein with reference to FIGS. 2 and 3A-3C. For example, the spargers can be spaced apart from one another along the straight wall length L of the fermentation vessel by a distance within 10% of D. Or, for example, the spargers can be spaced apart from one another along the straight wall length L of the fermentation vessel by a distance within 5% of D. Or, for example, the spargers can be spaced apart from one another along the straight wall length L of the fermentation vessel by a distance of D. In some configurations of fermentation method 400, the spargers are spaced unevenly from one another, e.g., in a manner such as described herein with reference to FIGS. 2 and 3A-3C, particularly FIG. 3B.

In some configurations of fermentation method 400, at least one of the spargers includes a double-ring sparger, e.g., in a manner such as described herein with reference to FIG. 2. In some configurations of fermentation method 400, introducing the gas includes introducing a first gas and a second gas, at least one of the first and second gases including the reactive gaseous component, e.g., in a manner such as described herein with reference to FIG. 2. Optionally, at least one of the spargers introduces bubbles including a mixture of the first gas and the second gas into the fermentation broth. As another option, at least one of the spargers introduces bubbles including a different mixture of the first gas and the second gas than does at least one other of the spargers. As yet another option, the first gas is air and the second gas is substantially pure oxygen.

In some configurations of fermentation method 400, the gas is air. In some configurations of fermentation method 400, the gas is substantially pure oxygen. In some configurations of fermentation method 400, the reactive gaseous component is selected from the group consisting of oxygen, methane, carbon monoxide, carbon dioxide, nitrogen, and hydrogen. For example, the reactive gaseous component optionally can be oxygen. Or, for example, the reactive gaseous component can be carbon dioxide.

In some configurations of fermentation method 400, the method further includes adjusting an introduction rate of the reactive gaseous component by at least one of the spargers as a function of time, e.g., in a manner such as described herein with reference to FIG. 2. For example, the method can include adjusting the introduction rate of the reactive gaseous component by each of the spargers as a function of time. Or, for example, the method can include, responsive to the adjustment of the introduction rate of the reactive gaseous component, a microbial organism in the fermentation broth favors a biological pathway producing a product. Optionally, the product can be selected from the group consisting of 1,4-butanediol, 1,3-butanediol, caprolactam, adipic acid, and 6-amino-caproic acid.

In some configurations of fermentation method 400, at least one of the spargers has a different introduction rate of the reactive gaseous component than does at least one other of the spargers, e.g., in a manner such as described herein with reference to FIG. 2. In some configurations of fermentation method 400, each of the spargers includes a ring sparger, e.g., in a manner such as described herein with reference to FIG. 2. In some configurations of fermentation method 400, at least one of the spargers includes a nozzle or pipe sparger, e.g., in a manner such as described herein with reference to FIG. 2. In some configurations of fermentation method 400, responsive to release of the reactive gaseous component within the gas, a microbial organism in the fermentation broth produces a product, e.g., in a manner such as described herein with reference to FIG. 2. Optionally, the product can be selected from the group consisting of 1,4-butanediol, 1,3-butanediol, caprolactam, adipic acid, and 6-amino-caproic acid. Additionally, or alternatively, the microbial organism can include a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Other microbial organisms that are genetically engineered so as to produce products can include a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, and Rhizopus oryzae. and Yarrowia lipolytica. Still other microbial organisms that are genetically engineered so as to produce products can include methanotrophs. Still other microbial organisms that are genetically engineered so as to produce products can include algae.

An exemplary problem solved by the present fermentation systems and methods is that of scaling up production of a product from a small, ideally mixed lab scale reactor to a large-scale bioreactor, e.g., a bubble column, such as suitable for generating relatively large volumes of product such as can be suitable for use in industrial processes. For example, a “small-scale” or “lab scale” fermentation vessel (bioreactor) may hold less than 10 L of fermentation broth, or at most about 10-50 L of fermentation broth. In comparison, a “large-scale” or “industrial scale” fermentation vessel (bioreactor) can hold 20,000 L or more of fermentation broth, e.g., 100,000 L or more, or 200,000 or more, or even 500,000 or more of fermentation broth. In large-scale bioreactors such as bubble columns, extended mixing times, combined with relatively uneven power distribution, hydrostatic pressure gradients, and dynamic gas phase composition can result in gradients in the VUR and gradients in mass transfer. For a fermentation process using gaseous substrates and/or nutrients, such as one or more reactive gaseous components, gradients in distribution and/or delivery of such component(s) can negatively impact performance of a microbial organism in the fermentation broth, particularly as scale increases. Reduced performance of the microbial organism can have significant cost implications for a manufacturing plant that is fermenting the microbial organism to produce a product.

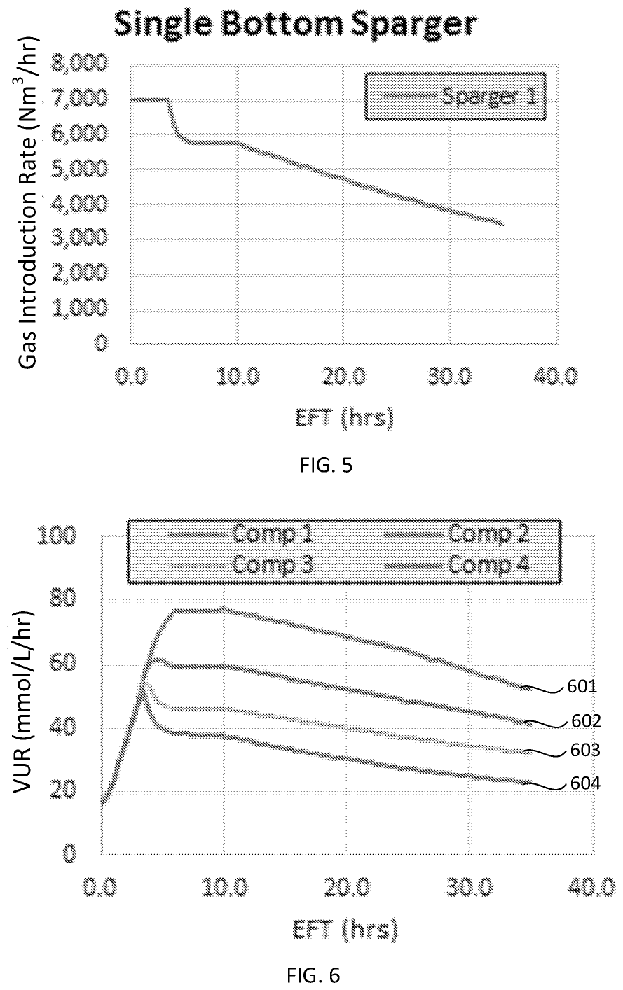

To further aid in the understanding the performance and results of the present fermentation systems and methods, exemplary data from simulations will be described with reference to FIGS. 5-13. It should be understood that such data is intended to be purely illustrative, and not limiting. FIG. 5 is a plot illustrating a simulated exemplary introduction rate of a gas in a fermentation system having a single sparger, e.g., fermentation system 100 illustrated in FIG. 1 having single sparger 110 introducing a gas including a reactive gaseous component. In one nonlimiting example, the gas is air, and the reactive gaseous component is oxygen (X_{g}=0.21). Total fermentor volume=620,000 L with a total L/D of 5.5 (D=5.3 m, L straight wall=26.5 m, L total vessel=29.1 m). Initially (t=0.0 hours), the introduction rate of the gas is approximately 7,000 Nm³/hour. Beginning at about t=4 hours, the introduction rate of the gas is gradually reduced to approximately 5,800 Nm³/hour, and then beginning at about t=10 hours, the introduction rate of the gas is gradually further reduced to about 3,500 Nm³/hour at about t=35 hours. Beginning the introduction rate of the gas at a relatively high level can cause the microorganism to favor a first metabolic pathway in which the microorganism readily grows and multiplies. After a period of such growth and reproduction, reducing the introduction rate of the gas can cause the (multiplied) microorganism to favor a second metabolic pathway in which the microorganism produces a product. Exemplary products include, but are not limited to, 1,4-butanediol, 1,3-butanediol, caprolactam, adipic acid, and 6-amino-caproic acid. As one example, E. coli reproduces and grows well at a relatively high introduction rate of air, and switches to favoring a product-producing pathway at a sufficiently low introduction rate of air.

As noted further above, use of a single sparger within a bubble column can create a significant gradient in VUR of a reactive gaseous component within the gas released by the sparger. FIG. 6 is a plot illustrating simulated VURs of a reactive gaseous component in different mixing zones a fermentation system having a single sparger installed at the bottom of the vessel, e.g., the sparger described above with reference to FIG. 5. More specifically, in this nonlimiting example, the VUR of the reactive gaseous component (such as oxygen in air introduced by the single sparger) was simulated at four different vertical levels within the simulated fermentation vessel. The average broth height over the fed-batch process was used to determine the number of compartments (n=4). The broth was then divided into four compartments of equal height, L, which changes some over the time course simulation as the reactor fills. The compartment VURs in FIG. 6 are the average values for each compartment, calculated using the average C* (P, Xg) and k_{L}a of each compartment (arithmetic average of the lower and upper level of each compartment). Values for C* and k_{L}a are solved simultaneously and iteratively as C* depends on k_{L}a and vice versa. It can be seen that the VUR 601 at a first (lowest) level within the fermentation vessel gradually increased to a level of about 78 mmol/L/hour beginning around t=6 hours, and then beginning at about t=10 hours gradually decreased to about 52 mmol/L/hour at around t=35 hours. It also can be seen that the VUR 602 at a second (second lowest) level within the fermentation vessel gradually increased to a level of about 60 mmol/L/hour beginning around t=6 hours, and then beginning at about t=10 hours gradually decreased to about 41 mmol/L/hour at around t=35 hours. It also can be seen that the VUR 603 at a third (second highest) level within the fermentation vessel gradually increased to a level of about 47 mmol/L/hour beginning around t=6 hours, and then beginning at about t=10 hours gradually decreased to about 32 mmol/L/hour at around t=35 hours. It also can be seen that the VUR 604 at a fourth (highest) level within the fermentation vessel gradually increased to a level of about 38 mmol/L/hour beginning around t=6 hours, and then beginning at about t=10 hours gradually decreased to about 23 mmol/L/hour at around t=35 hours. Accordingly, from FIG. 6 it can be understood that the VURs in the fermentation vessel at any given moment of time can vary significantly across the vessel (e.g., at about t=10 hours, by about 78 mmol/L/hour at the first level versus about 38 mmol/L/hour at the highest level, an approximately 205% difference). Additionally, from FIG. 6 it can be understood that the differences in VURs across the vessel also can change as a function of time (e.g., from the approximately 205% different at about t=10 hours, to a difference of about 52 mmol/L/hour at the first level versus about 23 mmol/L/hour at the highest level at about t=35 hours, an approximately 226% difference).

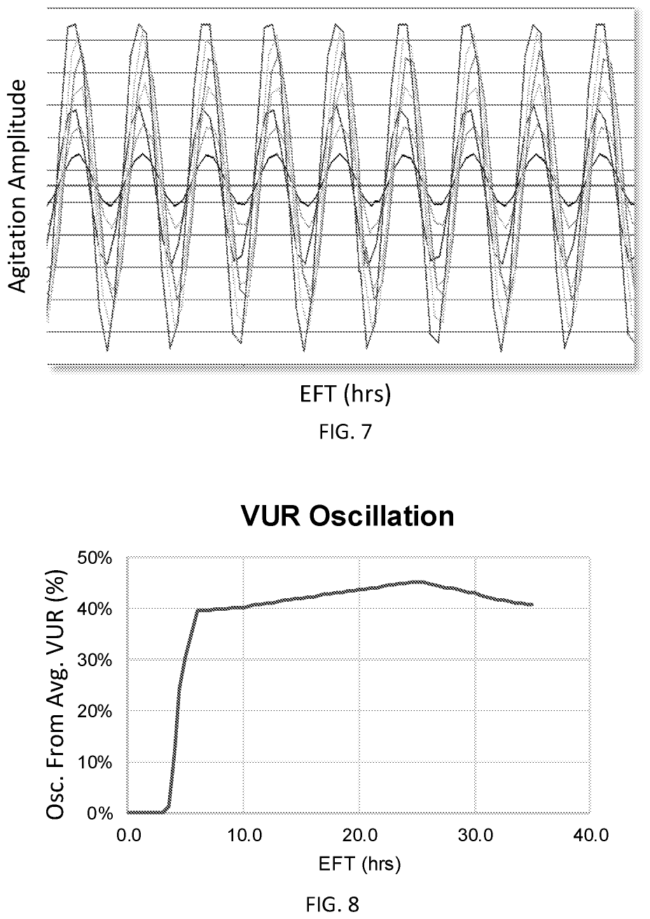

The simulations in FIGS. 6 and 10 were performed by modeling an oscillating mass transfer rate in a simulated 2 L mechanically agitated bioreactor under the condition that the percent of dissolved reactive gaseous component (the value C in Equation (1)) is equal to zero. Under such condition, Equation (1) can be expressed as VUR=k_{L}a×C*, and the VUR of the reactive gaseous component is equal to the VTR of the reactive gaseous component. The volumetric gas-liquid mass transfer coefficient (k_{L}a) is a function of power input per unit volume (P/V); in a lab reactor, most of the power delivery comes from the agitator, whereas in a bubble column all of the power delivery comes from release of bubbles. Dynamic manipulation of the agitation rate provides a simple means of changing the mass transfer characteristics (VTR), which changes the VUR; oscillating on a time scale equivalent to the expected mixing time at large scale (e.g., 60-180 seconds) allows for simulation of gradients. For example, FIG. 7 is a plot illustrating oscillations in agitation of varying magnitude to simulate a gradient in VUR of a reactive gaseous component in a fermentation system. FIG. 8 is a plot illustrating a simulated percent oscillation from an average VUR of a reactive gaseous component in a fermentation system having a single sparger installed at the bottom of the vessel. More specifically, FIG. 7 illustrates actual lab data from bioreactor experiments with a custom controller that dynamically adjusted the stirrer agitation rate (rpm) to change k_{L}a, and hence VTR of oxygen. FIG. 8 corresponds to the model simulation in FIG. 6, but depicts the % oscillation in VUR from the total vessel average. For example, at EFT 10 hrs, the average VUR is about 55 mmol/L/h, max VUR is about 77 mmol/L/h (comp1, 601), min VUR is about 33 mmol/L/h (comp4, 604); thus, the oscillation is 55+/−22 mmol/L/h; 22/55=40%, which corresponds to the % oscillation depicted at 10 hrs in FIG. 8.

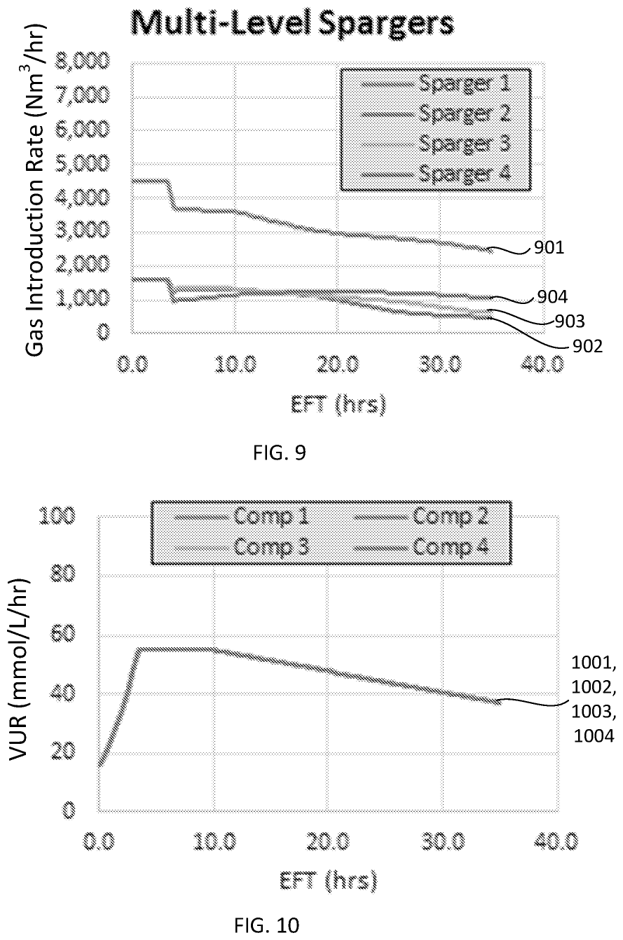

As provided herein, fermentation systems and methods that include sparging at multiple vertical levels within a fermentation vessel, such as a bubble column reactor, can significantly reduce differences/gradients in the VUR across the length of the vessel. For example, FIG. 9 is a plot illustrating an exemplary introduction rates of a gas in a fermentation system having a multiple spargers, according to some configurations provided herein, e.g., fermentation system 200 illustrated in FIG. 2, including fermentation vessel 210, 310, 310′, or 310″. In FIG. 9, the fermentation system of FIGS. 5-6 was simulated to include four spargers. the spargers are at the bottom of each compartment described with reference to FIGS. 5-6; the values in the graphs are averages for each compartment (average of the bottom level at the sparger and top of compartment). This simulation was done using the same method as outlined above; however, for this simulation, because the sparger locations are fixed, the dimensions of compartments 1-3 are static (L doesn't change over fermentation time course) and only the L of compartment 4 changes over time as the fermentor is filled. The sparger spacing was calculated by dividing the average L of the broth over the entire time course by the number of compartments. With this spacing, the top sparger was submerged for the entire time course. FIG. 9 illustrates that the gas introduction rate at each of these spargers was varied differently than one another as a function of time. For example, at the first location, the gas introduction rate 901 initially (t=0.0 hours) is approximately 4,500 Nm³/hour; is reduced to approximately 3,600 Nm³/hour beginning at about t=4 hours; and then beginning at about t=10 hours, the introduction rate of the gas is gradually further reduced to about 2,400 Nm³/hour at about t=35 hours. At the second location, the gas introduction rate 902 initially (t=0.0 hours) is approximately 1,600 Nm³/hour; is reduced to approximately 1,300 Nm³/hour beginning at about t=4 hours; and then beginning at about t=15 hours, the introduction rate of the gas is gradually further reduced to about 500 Nm³/hour at about t=35 hours. At the third location, the gas introduction rate 903 initially (t=0.0 hours) is approximately 1,600 Nm³/hour; is reduced to approximately 1,400 Nm³/hour beginning at about t=4 hours; and then beginning at about t=15 hours, the introduction rate of the gas is gradually further reduced to about 600 Nm³/hour at about t=35 hours. At the fourth location, the gas introduction rate 904 initially (t=0.0 hours) is approximately 1,600 Nm³/hour; is reduced to approximately 1,000 Nm³/hour beginning at about t=4 hours; and then beginning at about t=5 hours, the introduction rate of the gas is gradually increased to about 1,400 Nm³/hour at about t=15 hours before being gradually decreased to about 1,000 Nm³/hour at about t=35 hours.

As provided herein, suitably selecting the respective flows of gas(es) including a reactive gaseous component through suitably located spargers can significantly reduce or eliminate differences or gradients in the VUR of the reactive gaseous component within a fermentation vessel. For example, FIG. 10 is a plot illustrating exemplary VUR of a reactive gaseous component in a fermentation system having a multiple spargers, according to some configurations provided herein. More specifically, FIG. 10 is a plot of the respectively simulated VUR 1001, 1002, 1003, 1004 at the first, second, third, and fourth locations in the fermentation vessel simulated in FIG. 9. It may be understood from FIG. 10 that the VUR at each of the four locations is substantially the same as one another.

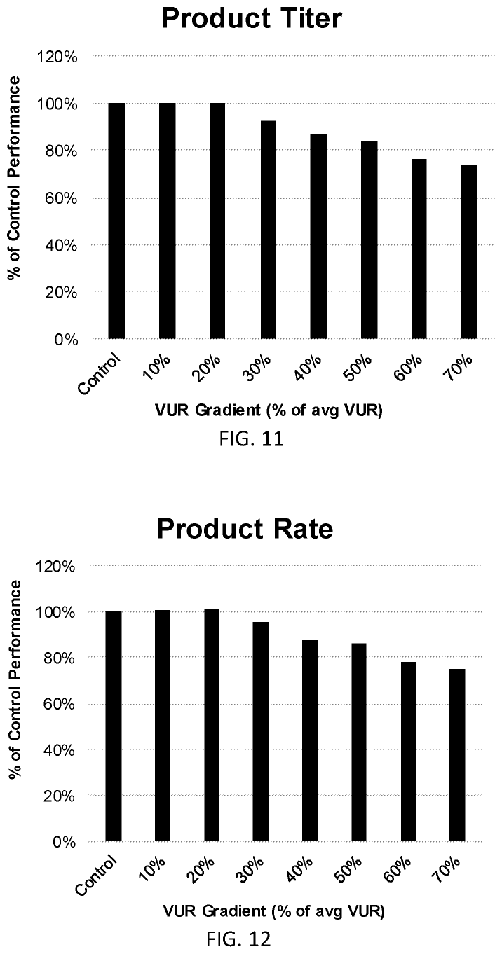

Additionally, as provided herein, reducing or eliminating differences or gradients in the VUR of a reactive gaseous component within the present fermentation systems and methods can improve production of a product by a microorganism. For example, FIG. 11 is a plot illustrating product titer as a function of VUR gradient, according to some configurations provided herein. The data shown in FIGS. 11-13 were obtained using laboratory fermentations conducted in 2 L bioreactors. Oxygen VUR variability was induced by oscillating the stirrer agitation rate to alter the mass transfer rate of oxygen, thus simulating a gradient in VUR. In FIG. 11, the titers of a product at VUR gradients (% of average VUR) of between 10-70% are normalized against that of an ideal “control” VUR profile having no variation. It can be understood in FIG. 11 that the product titer for a VUR gradient of about 10% is about 100% of the control performance—comparable to that of the control VUR, and that the product titer for a VUR gradient of about 20% is about 98% of the control performance—again comparable to that of the control VUR. However, for greater VUR gradients, the product titer can be understood to decrease. For example, the product titer for a VUR gradient of about 30% is about 93% of the control performance; the product titer for a VUR gradient of about 40% is about 87% of the control performance; the product titer for a VUR gradient of about 50% is about 82% of the control performance; the product titer for a VUR gradient of about 60% is about 78% of the control performance; and the product titer for a VUR gradient of about 70% is about 75% of the control performance. Accordingly, it can be understood from FIG. 11 that VUR gradients of greater than about 20% can detrimentally impact product titer, and that VUR gradients of about 20% or less perform comparably to the control process and do not reduce product titer.

FIG. 12 is a plot illustrating product rate as a function of VUR gradient, according to some configurations provided herein. In FIG. 12, the product rate at VUR gradients (% of average VUR) of between 10-70% are normalized against that of an ideal “control” VUR profile having no variation. It can be understood in FIG. 12 that the product rate for a VUR gradient of about 10% is about 102% of the control performance, and that the product rate for a VUR gradient of about 20% is about 104% of the control performance—both of which are comparable to and even better that of the control VUR. Note that typical variation for product rate is about 2-3%; for example, several factors than can influence the rate of a fermentation process (e.g. any deviation in VUR, the amount of substrate fed, the total time of the fermentation batch, and the like). Accordingly, in some circumstances the product rate potentially can exceed that of the control. However, for greater VUR gradients, the product rate can be understood to decrease. For example, the product rate for a VUR gradient of about 30% is about 95% of the control performance; the product rate for a VUR gradient of about 40% is about 86% of the control performance; the product rate for a VUR gradient of about 50% is about 82% of the control performance; the product rate for a VUR gradient of about 60% is about 78% of the control performance; and the product rate for a VUR gradient of about 70% is about 75% of the control performance. Accordingly, it can be understood from FIG. 12 that VUR gradients of greater than about 20% can detrimentally impact product rate, and that VUR gradients of about 20% or less perform comparably to the control process and do not reduce product rate.

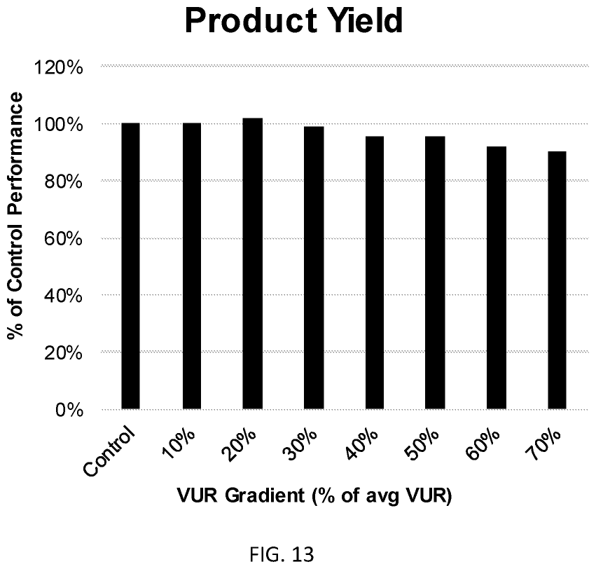

FIG. 13 is a plot illustrating product yield as a function of VUR gradient, according to some configurations provided herein. In FIG. 13, the product yield at VUR gradients (% of average VUR) of between 10-70% are normalized against that of an ideal “control” VUR profile having no variation. It can be understood in FIG. 13 that the product yield for a VUR gradient of about 10% is about 100% of the control performance, and that the product yield for a VUR gradient of about 20% is about 104% of the control performance—performance—both of which are comparable to and even better that of the control VUR for similar reasons as explained with reference to FIG. 12. However, for greater VUR gradients, the product yield can be understood to decrease. For example, the product yield for a VUR gradient of about 30% is about 98% of the control performance; the product yield for a VUR gradient of about 40% is about 94% of the control performance; the product yield for a VUR gradient of about 50% is about 94% of the control performance; the product yield for a VUR gradient of about 60% is about 92% of the control performance; and the product yield for a VUR gradient of about 70% is about 91% of the control performance. Accordingly, it can be understood from FIG. 13 that VUR gradients of greater than about 20% can detrimentally impact product yield, and that VUR gradients of about 20% or less perform comparably to the control process and do not reduce product yield.

As provided herein, the present fermentation systems and methods can provide VURs that vary by no more than about 20% along the length of the fermentation vessel, or no more than about 10% along the length of the fermentation vessel, or no more than about 5% along the length of the fermentation vessel. Accordingly, product titers, product rates, and product yields similar to that of an idea control having no VUR gradient can be expected, such as may be understood from FIGS. 11-13.

Genetic Alteration of Microbes/Orthologs/Paralogs

Non-naturally occurring microbial organisms that can be used with the present fermentation systems and methods can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing non-naturally occurring microbial organisms having product biosynthetic capability, such as 1,4-butanediol, 1,3-butanediol, caprolactam, adipic acid, or 6-amino-caproic acid biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In an additional configuration, the present fermentation systems and methods can be used with a non-naturally occurring microbial organism having a product pathway, such as a 1,4-butanediol, 1,3-butanediol, caprolactam, adipic acid, or 6-amino-caproic acid pathway, wherein the non-naturally occurring microbial organism includes at least one exogenous nucleic acid encoding an enzyme or protein that converts a substrate (such as a reactive gaseous component) to the product via suitable intermediates. One skilled in the art will understand any substrate-product pairs suitable to produce a desired product and for which an appropriate activity is available for the conversion of the substrate to the product can be readily determined by one skilled in the art based on the teachings herein. While generally described herein as a microbial organism that contains a product pathway, it is understood that present fermentation systems and methods also or alternatively can be used with a non-naturally occurring microbial organism including at least one exogenous nucleic acid encoding a product pathway enzyme or protein expressed in a sufficient amount to produce an intermediate of a product pathway. Furthermore, a microbial organism that produces an intermediate can be used in combination with another microbial organism expressing downstream pathway enzymes to produce a desired product. However, it is understood that a non-naturally occurring microbial organism that produces a product pathway intermediate can be utilized to produce the intermediate as a desired product.

Metabolic Reactions

The present fermentation systems and methods are described herein with general reference to reaction of the gaseous reactive component, which can include the metabolic reaction, reactant or product thereof, or one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction (the gaseous reactive component can be one of such reactants). Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

Host Microbes

The non-naturally occurring microbial organisms that can be used with the present fermentation systems and methods can be produced by introducing expressible nucleic acids encoding one or more of the enzymes or proteins participating in one or more product pathways, such as one or more 1,4-butanediol, 1,3-butanediol, caprolactam, adipic acid, or 6-amino-caproic acid biosynthetic pathways. Depending on the host microbial organism chosen for biosynthesis, nucleic acids for some or all of a particular biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve product biosynthesis. Thus, a non-naturally occurring microbial organism suitable for use in the present fermentation systems and methods can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as 1,4-butanediol, 1,3-butanediol, caprolactam, adipic acid, or 6-amino-caproic acid.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. Other exemplary microbial organisms suitable for use in the present fermentation systems and methods include methanotrophs. Still other exemplary microbial organisms suitable for use in the present fermentation systems include algae. E. coli is a particularly useful host organisms since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the product biosynthetic pathway constituents of a selected host microbial organism, the non-naturally occurring microbial organisms suitable for use in the present fermentation systems and methods will include at least one exogenously expressed product pathway-encoding nucleic acid and up to all encoding nucleic acids for one or more product biosynthetic pathways. For example, product biosynthesis can be established in a host deficient in a pathway enzyme or protein through exogenous expression of the corresponding encoding nucleic acid. In a host deficient in all enzymes or proteins of a product pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of the product, such as 1,4-butanediol, 1,3-butanediol, caprolactam, adipic acid, or 6-amino-caproic acid, can be included.

Given the teachings and guidance provided herein, those skilled in the art will understand that the number of encoding nucleic acids to introduce in an expressible form will, at least, parallel the product pathway deficiencies of the selected host microbial organism. Therefore, a non-naturally occurring microbial organism suitable for use in the present fermentation systems and methods can have one, two, three, four, or any suitable number, up to all nucleic acids encoding the enzymes or proteins constituting a product biosynthetic pathway. In some configurations, the non-naturally occurring microbial organisms also can include other genetic modifications that facilitate or optimize product biosynthesis or that confer other useful functions onto the host microbial organism. One such other functionality can include, for example, augmentation of the synthesis of one or more of the product pathway precursors.

Generally, a host microbial organism is selected such that it produces the precursor of a product pathway, such as a 1,4-butanediol, 1,3-butanediol, caprolactam, adipic acid, or 6-amino-caproic acid pathway, either as a naturally produced molecule or as an engineered product that either provides de novo production of a desired precursor or increased production of a precursor naturally produced by the host microbial organism. For example, certain precursors such as succinate are produced naturally in a host organism such as E. coli. A host organism can be engineered to increase production of a precursor. In addition, a microbial organism that has been engineered to produce a desired precursor can be used as a host organism and further engineered to express enzymes or proteins of a product pathway, such as a 1,4-butanediol, 1,3-butanediol, caprolactam, adipic acid, or 6-amino-caproic acid pathway.

In some configurations, a non-naturally occurring microbial organism suitable for use in the present fermentation systems and methods is generated from a host that contains the enzymatic capability to synthesize the product, such as 1,4-butanediol, 1,3-butanediol, caprolactam, adipic acid, or 6-amino-caproic acid. In this specific configuration it can be useful to increase the synthesis or accumulation of product pathway product to, for example, drive pathway reactions toward production of the product. Increased synthesis or accumulation can be accomplished by, for example, overexpression of nucleic acids encoding one or more of the above-described pathway enzymes or proteins. Overexpression the enzyme or enzymes and/or protein or proteins of the product pathway can occur, for example, through exogenous expression of the endogenous gene or genes, or through exogenous expression of the heterologous gene or genes. In addition, a non-naturally occurring organism can be generated by mutagenesis of an endogenous gene that results in an increase in activity of an enzyme in the 1,4-butanediol, 1,3-butanediol, caprolactam, adipic acid, or 6-amino-caproic acid biosynthetic pathway.

In particularly useful configurations, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other configurations such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in the present fermentation systems and methods, any of the one or more exogenous nucleic acids can be introduced into a microbial organism to produce a non-naturally occurring microbial organism suitable for use therein. The nucleic acids can be introduced so as to confer, for example, a product biosynthetic pathway onto the microbial organism. Alternatively, encoding nucleic acids can be introduced to produce an intermediate microbial organism having the biosynthetic capability to catalyze some of the required reactions to confer product biosynthetic capability. For example, a non-naturally occurring microbial organism having a product biosynthetic pathway can include at least two exogenous nucleic acids encoding desired enzymes or proteins. Thus, it is understood that any combination of two or more enzymes or proteins of a biosynthetic pathway can be included in a non-naturally occurring microbial organism suitable for use in the present fermentation systems and methods. Similarly, it is understood that any combination of three or more enzymes or proteins of a biosynthetic pathway can be included in a non-naturally occurring microbial organism suitable for use in the present fermentation systems and methods, so long as the combination of enzymes and/or proteins of the desired biosynthetic pathway results in production of the corresponding desired product. Similarly, any combination of four or more enzymes or proteins of a biosynthetic pathway can be included in a non-naturally occurring microbial organism, as desired, so long as the combination of enzymes and/or proteins of the desired biosynthetic pathway results in production of the corresponding desired product.

In addition to the biosynthesis of products, such as 1,4-butanediol, 1,3-butanediol, caprolactam, adipic acid, or 6-amino-caproic acid, the non-naturally occurring microbial organisms suitable for use in the present fermentation systems and methods also can be utilized in various combinations with each other and with other microbial organisms and methods well known in the art to achieve product biosynthesis by other routes. For example, one alternative to produce a product other than use of the product producers is through addition of another microbial organism capable of converting a product pathway intermediate to the product. One such procedure includes, for example, the fermentation of a microbial organism that produces a product pathway intermediate. The product pathway intermediate can then be used as a substrate for a second microbial organism that converts the product pathway intermediate to the product, such as 1,4-butanediol, 1,3-butanediol, caprolactam, adipic acid, or 6-amino-caproic acid. The product pathway intermediate can be added directly to another culture of the second organism or the original culture of the product pathway intermediate producers can be depleted of these microbial organisms by, for example, cell separation, and then subsequent addition of the second organism to the fermentation broth can be utilized to produce the final product without intermediate purification steps.

In other configurations, the non-naturally occurring microbial organisms can be assembled in a wide variety of subpathways to achieve biosynthesis of the product, for example, 1,4-butanediol, 1,3-butanediol, caprolactam, adipic acid, or 6-amino-caproic acid. In these configurations, biosynthetic pathways for a desired product within the present fermentation systems and methods can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of the product, such as 1,4-butanediol, 1,3-butanediol, caprolactam, adipic acid, or 6-amino-caproic acid, can be accomplished by constructing a microbial organism that contains biosynthetic pathways for conversion of one pathway intermediate to another pathway intermediate or the product. Alternatively, the product also can be biosynthetically produced from microbial organisms through co-culture or co-fermentation using two organisms in the same vessel, where the first microbial organism produces a product intermediate and the second microbial organism converts the intermediate to the product, such as 1,4-butanediol, 1,3-butanediol, caprolactam, adipic acid, or 6-amino-caproic acid.

Given the teachings and guidance provided herein, those skilled in the art will understand that a wide variety of combinations and permutations exist for the non-naturally occurring microbial organisms together with other microbial organisms, with the co-culture of other non-naturally occurring microbial organisms having subpathways and with combinations of other chemical and/or biochemical procedures well known in the art to produce a product, such as 1,4-butanediol, 1,3-butanediol, caprolactam, adipic acid, or 6-amino-caproic acid.

Source of Genes/Host Organisms

Sources of encoding nucleic acids for a product pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite product biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of products such as 1,4-butanediol, 1,3-butanediol, caprolactam, adipic acid, or 6-amino-caproic acid described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative product biosynthetic pathway exists in an unrelated species, product biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods herein can be applied to all microbial organisms using the cognate metabolic alterations to those exemplified herein to construct a microbial organism in a species of interest that will synthesize the product, such as 1,4-butanediol, 1,3-butanediol, caprolactam, adipic acid, or 6-amino-caproic acid.

Construction of Microbes/Testing Expression

Methods for constructing and testing the expression levels of a non-naturally occurring product-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of a product, such as 1,4-butanediol, 1,3-butanediol, caprolactam, adipic acid, or 6-amino-caproic acid, can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more product biosynthetic pathway encoding nucleic acids as exemplified herein operably linked to expression control sequences functional in the host organism. Expression vectors applicable for use in the microbial host organisms for use in the present fermentation systems and methods include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression.

Suitable purification and/or assays to test for the production of a product, such as 1,4-butanediol, 1,3-butanediol, caprolactam, adipic acid, or 6-amino-caproic acid, can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

Separation/Purification Techniques

The product, such as 1,4-butanediol, 1,3-butanediol, caprolactam, adipic acid, or 6-amino-caproic acid, can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Growth Media/Conditions

Any of the non-naturally occurring microbial organisms described herein can be cultured to produce and/or secrete the biosynthetic products in the present fermentation systems and methods. For example, the 1,4-butanediol, 1,3-butanediol, caprolactam, adipic acid, or 6-amino-caproic acid producers can be cultured for the biosynthetic production of those respective products.

For the production of products, such as 1,4-butanediol, 1,3-butanediol, caprolactam, adipic acid, or 6-amino-caproic acid, the recombinant strains are cultured in the present fermentation vessel (such as vessel 210, 310, 310′, or 310″) in a medium (fermentation broth) with carbon source and other essential nutrients. It is sometimes desirable and can be highly desirable to maintain anaerobic conditions in the fermentation vessel to reduce the cost of the overall process. Such conditions can be obtained, for example, by first sparging the medium with nitrogen and then sealing the fermentation vessel. For strains where growth is not observed anaerobically, aerobic or substantially anaerobic conditions can be applied by releasing air, oxygen, or any suitable oxygen-containing mixture(s) using the present spargers, for limited aeration. Exemplary anaerobic conditions have been described previously and are well-known in the art. Exemplary aerobic and anaerobic conditions are described, for example, in United States publication 2009/0047719, filed Aug. 10, 2007. Fermentations can be performed in a batch, fed-batch or continuous manner.

If desired, the pH of the medium can be maintained at a desired pH, in particular neutral pH, such as a pH of around 7 by addition of a base, such as ammonia, NaOH or other bases, or acid, as needed to maintain the culture medium at a desirable pH. Additionally, as noted above, the pH in each of the present mixing zones can be monitored by a suitable probe, and controlled by inputting a suitable pH adjustant via the sparger corresponding to that mixing zone. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the present fermentation systems and methods include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms for the production of a product, such as 1,4-butanediol, 1,3-butanediol, caprolactam, adipic acid, or 6-amino-caproic acid, in the present fermentation systems and methods.

In addition to renewable feedstocks such as those exemplified above, the microbial organisms also or alternatively can be modified for growth on syngas as its source of carbon. In this specific configuration, one or more proteins or enzymes are expressed in the product producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂. As noted above, hydrogen, carbon monoxide, and carbon dioxide suitably can be used as reactive gaseous components in some configurations of the present fermentation systems and methods.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP (2)

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a product pathway, such as a 1,4-butanediol, 1,3-butanediol, caprolactam, adipic acid, or 6-amino-caproic acid pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms such that the modified organism contains the complete Wood-Ljungdahl pathway will confer syngas utilization ability in the present fermentation systems and methods.

Additionally, the reductive (reverse) tricarboxylic acid cycle is and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the product precursors, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a product pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms such that the modified organism contains the complete reductive TCA pathway will confer syngas utilization ability within the present fermentation systems and methods.

Accordingly, given the teachings and guidance provided herein, those skilled in the art will understand that a non-naturally occurring microbial organism can be produced that secretes the biosynthesized product in the present fermentation vessels and methods when grown on a carbon source such as a carbohydrate. Such compounds include, for example, 1,4-butanediol, 1,3-butanediol, caprolactam, adipic acid, or 6-amino-caproic acid, and any of the intermediate metabolites in those product pathways. All that is required is to engineer in one or more of the required enzyme or protein activities to achieve biosynthesis of the desired compound or intermediate including, for example, inclusion of some or all of the product biosynthetic pathways. The non-naturally occurring microbial organisms can be constructed using methods well known in the art as exemplified herein to exogenously express at least one nucleic acid encoding a product pathway enzyme or protein in sufficient amounts to produce the product. It is understood that the microbial organisms are cultured under conditions sufficient to produce the product within the present fermentation systems and methods. Following the teachings and guidance provided herein, the non-naturally occurring microbial organisms can achieve biosynthesis of the product resulting in intracellular concentrations between about 0.1-2000 mM or more. In some configurations, the intracellular concentration of the product is between about 300-1500 mM, particularly between about 500-1250 mM and more particularly between about 800-1000 mM, or more. Intracellular concentrations between and above each of these exemplary ranges also can be achieved from the non-naturally occurring microbial organisms within the present fermentation systems and methods. In some configurations, a product (such as, but not limited to, 1,4-butanediol or 1,3-butanediol) can freely diffuse across the membrane of the cell, which means intracellular product concentration will be as high as the extracellular (e.g., 500 mM or more, or 1000 mM or more, or 1500 mM or more).

In some configurations, culture conditions include anaerobic or substantially anaerobic growth or maintenance conditions. Exemplary anaerobic conditions have been described previously and are well known in the art and can be achieved by releasing gas(es) of appropriate composition(s) through respective spargers in the present fermentation systems and methods. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the product producers can synthesize a product, such as 1,4-butanediol, 1,3-butanediol, caprolactam, adipic acid, or 6-amino-caproic acid, at intracellular concentrations of 5-10 mM or more as well as all other concentrations exemplified herein. It is understood that product producing microbial organisms can produce the product intracellularly and/or secrete the product into the culture medium.

In addition to the culturing and fermentation conditions disclosed herein, growth condition for achieving biosynthesis of the product can include the addition of an osmoprotectant to the culturing conditions. In certain configurations, the non-naturally occurring microbial organisms can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethyl sulfonio-2-methylproprionate, pipecolic acid, dimethyl sulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

Growth/Fermentation Conditions

The culture conditions can include, for example, liquid culture procedures as well as fermentation and other large scale culture procedures. As described herein, particularly useful yields of certain biosynthetic products can be obtained under anaerobic or substantially anaerobic culture conditions in the present fermentation systems and methods, while yields of other biosynthetic products can be obtained under aerobic culture conditions in the present fermentation systems and methods. Exemplary reactive gaseous components can include, but are not limited to, oxygen, methane, carbon monoxide, carbon dioxide, nitrogen, and hydrogen.

For example, as described herein, one exemplary growth condition for achieving biosynthesis of a product includes anaerobic culture or fermentation conditions. In certain configurations, the non-naturally occurring microbial organisms can be sustained, cultured or fermented under anaerobic or substantially anaerobic conditions. Briefly, anaerobic conditions refers to an environment devoid of oxygen. In such anaerobic conditions, the reactive gaseous component can include, but is not limited to, methane, carbon monoxide, carbon dioxide, nitrogen or hydrogen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases using the spargers of the present fermentation system. In a substantially anaerobic condition, the reactive gaseous component can include oxygen, optionally in combination with another reactive gaseous component, such as methane, carbon monoxide, carbon dioxide, nitrogen, or hydrogen. In an aerobic condition, the reactive gaseous component can include oxygen, optionally in combination with another reactive gaseous component, such as methane, carbon monoxide, carbon dioxide, nitrogen, or hydrogen. As compared with a substantially anaerobic condition, the aerobic condition can use a substantially higher proportion of oxygen as the reactive gaseous component.

The culture conditions described herein can be scaled up and grown continuously for manufacturing of a product, such as 1,4-butanediol, 1,3-butanediol, caprolactam, adipic acid, or 6-amino-caproic acid. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of products such as 1,4-butanediol, 1,3-butanediol, caprolactam, adipic acid, or 6-amino-caproic acid. Generally, and as with non-continuous culture procedures, the continuous and/or near-continuous production of products will include culturing a non-naturally occurring product producing organism in the present fermentation systems and methods in sufficient nutrients and medium to sustain and/or nearly sustain growth in an exponential phase. Continuous culture under such conditions can be include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of a product, such as 1,4-butanediol, 1,3-butanediol, caprolactam, adipic acid, or 6-amino-caproic acid, can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the product producers for continuous production of substantial quantities of product, the producers also can be, for example, simultaneously subjected to chemical synthesis procedures to convert the product to other compounds or the product can be separated from the fermentation culture and sequentially subjected to chemical conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of a product, such as 1,4-butanediol, 1,3-butanediol, caprolactam, adipic acid, or 6-amino-caproic acid.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. The OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

A nucleic acid encoding a desired activity of a product pathway can be introduced into a host organism. In some cases, it can be desirable to modify an activity of a product pathway enzyme or protein to increase production of the product. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Often and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a product pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

Throughout this application various publications have been referenced. The disclosures of these publications in their entireties, including any GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

What is claimed:

1. A fermentation system, comprising:
a fermentation vessel having a straight wall length L and an inner diameter D;
a source of a gas comprising a reactive gaseous component;
spargers spaced apart from one another along the straight wall length L of the fermentation vessel and configured to introduce bubbles of the gas into fermentation broth within the fermentation vessel;
wherein the release of the bubbles of the gas by each of the spargers establishes a respective mixing zone within the fermentation broth within the fermentation vessel, and
wherein each mixing zone has substantially the same volumetric uptake rate of the reactive gaseous component by the fermentation broth as each other mixing zone.

2. The fermentation system of claim 1, wherein each mixing zone comprises an upflow region and a downflow region each established by release of the bubbles of the gas from the respective sparger.

3. The fermentation system of claim 1 or claim 2, wherein in at least one mixing zone, the volumetric uptake rate of the reactive gaseous component is limited by availability of the reactive gaseous component.

4. The fermentation system of any one of claims 1-3, wherein the volumetric uptake rate of the reactive gaseous component by the fermentation broth varies by 20% or less across the entire volume of the fermentation broth.

5. The fermentation system of any one of claims 1-4, wherein the volumetric uptake rate of the reactive gaseous component by the fermentation broth varies by 10% or less across the entire volume of the fermentation broth.

6. The fermentation system of any one of claims 1-5, wherein the volumetric uptake rate of the reactive gaseous component by the fermentation broth varies by 5% or less across the entire volume of the fermentation broth.

7. The fermentation system of any one of claims 1-6, wherein each mixing zone has a volumetric uptake rate of the reactive gaseous component within 20% of that of each other mixing zone.

8. The fermentation system of any one of claims 1-7, wherein each mixing zone has a volumetric uptake rate of the reactive gaseous component within 10% of that of each other mixing zone.

9. The fermentation system of any one of claims 1-8, wherein each mixing zone has a volumetric uptake rate of the reactive gaseous component within 5% of that of each other mixing zone.

10. The fermentation system of any one of claims 1-9, wherein the fermentation vessel comprises a bubble column reactor in which substantially all mixing of the fermentation broth is accomplished by release of the bubbles of the gas by the spargers.

11. The fermentation system of any one of claims 1-10, comprising three or more spargers.

12. The fermentation system of any one of claims 1-11, wherein L is equal to or greater than 2D.

13. The fermentation system of claim 12, comprising a number of spargers equal to L/D rounded up or down to an integer number.

14. The fermentation system of claim 13, wherein the spargers are spaced apart from one another along the straight wall length L of the fermentation vessel by a distance within 20% of D.

15. The fermentation system of claim 13, wherein the spargers are spaced apart from one another along the straight wall length L of the fermentation vessel by a distance within 10% of D.

16. The fermentation system of claim 13, wherein the spargers are spaced apart from one another along the straight wall length L of the fermentation vessel by a distance within 5% of D.

17. The fermentation system of claim 15, wherein the spargers are spaced apart from one another along the straight wall length L of the fermentation vessel by a distance of D.

18. The fermentation system of any one of claims 1-17, wherein at least one of the spargers comprises a double-ring sparger.

19. The fermentation system of any one of claims 1-18, wherein the source comprises respective sources of a first gas and a second gas, at least one of the first and second gases comprising the reactive gaseous component.

20. The fermentation system of claim 19, wherein at least one of the spargers is configured to introduce bubbles including a mixture of the first gas and the second gas into the fermentation broth.

21. The fermentation system of any one of claims 19-20, wherein at least one of the spargers is configured to introduce bubbles including a different mixture of the first gas and the second gas than does at least one other of the spargers.

22. The fermentation system of any one of claims 19-20, wherein the first gas is air and the second gas is substantially pure oxygen.

23. The fermentation system of any one of claims 1-18, wherein the gas is air.

24. The fermentation system of any one of claims 1-18, wherein the gas is substantially pure oxygen.

25. The fermentation system of any one of claims 1-24, wherein the reactive gaseous component is selected from the group consisting of oxygen, methane, carbon monoxide, carbon dioxide, nitrogen, and hydrogen.

26. The fermentation system of claim 25, wherein the reactive gaseous component is oxygen.

27. The fermentation system of claim 25, wherein the reactive gaseous component is carbon dioxide.

28. The fermentation system of any one of claims 1-27, further comprising a controller configured to adjust an introduction rate of the reactive gaseous component by at least one of the spargers as a function of time.

29. The fermentation system of claim 28, wherein the controller is configured to adjust the introduction rate of the reactive gaseous component by each of the spargers as a function of time.

30. The fermentation system of claim 28, wherein responsive to the adjustment of the introduction rate of the reactive gaseous component, a microbial organism in the fermentation broth favors a biological pathway producing a product.

31. The fermentation system of claim 30, wherein the product is selected from the group consisting of 1,4-butanediol, 1,3-butanediol, caprolactam, adipic acid, and 6-amino-caproic acid.

32. The fermentation system of any one of claims 1-31, wherein at least one of the spargers has a different introduction rate of the reactive gaseous component than does at least one other of the spargers.

33. The fermentation system of any one of claims 1-32, wherein each of the spargers comprises a ring sparger.

34. The fermentation system of any one of claims 1-32, wherein at least one of the spargers comprises a nozzle or pipe sparger.

35. The fermentation system of any one of claims 1-34, wherein responsive to release of the reactive gaseous component within the bubbles of the gas, a microbial organism in the fermentation broth produces a product.

36. The fermentation system of claim 35, wherein the product is selected from the group consisting of 1,4-butanediol, 1,3-butanediol, caprolactam, adipic acid, and 6-amino-caproic acid.

37. The fermentation system of any one of claims 35-36, wherein the microbial organism comprises a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

38. The fermentation system of any one of claims 35-36, wherein the microbial organism comprises a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and Yarrowia lipolytica.

39. The fermentation system of any one of claims 35-36, wherein the microbial organism comprises algae or a methanotroph.

40. A fermentation method, comprising:
providing a fermentation broth within a fermentation vessel having a straight wall length L and an inner diameter D; and
introducing bubbles of a gas into the fermentation broth by spargers spaced apart from one another along the straight wall length L of the fermentation vessel,
wherein the gas comprises a reactive gaseous component,
wherein the release of the bubbles of the gas by each of the spargers establishes a respective mixing zone within the fermentation broth within the fermentation vessel, and
wherein each mixing zone has substantially the same volumetric uptake rate of the reactive gaseous component by the fermentation broth as each other mixing zone.

41. The fermentation method of claim 40, wherein in at least one mixing zone, the volumetric uptake rate of the reactive gaseous component is limited by availability of the reactive gaseous component.

42. The fermentation method of claim 40 or 41, wherein each mixing zone comprises an upflow region and a downflow region each established by release of the bubbles of the gas from the respective sparger.

43. The fermentation method of any one of claims 40-42, wherein the volumetric uptake rate of the reactive gaseous component by the fermentation broth varies by 20% or less across the entire volume of the fermentation broth.

44. The fermentation method of any one of claims 40-43, wherein the volumetric uptake rate of the reactive gaseous component by the fermentation broth varies by 10% or less across the entire volume of the fermentation broth.

45. The fermentation method of any one of claims 40-44, wherein the volumetric uptake rate of the reactive gaseous component by the fermentation broth varies by 5% or less across the entire volume of the fermentation broth.

46. The fermentation method of any one of claims 40-45, wherein each mixing zone has a volumetric uptake rate of the reactive gaseous component within 20% of that of each other mixing zone.

47. The fermentation method of any one of claims 40-46, wherein each mixing zone has a volumetric uptake rate of the reactive gaseous component within 10% of that of each other mixing zone.

48. The fermentation method of any one of claims 40-47, wherein each mixing zone has a volumetric uptake rate of the reactive gaseous component within 5% of that of each other mixing zone.

49. The fermentation method of any one of claims 40-48, wherein the fermentation vessel comprises a bubble column reactor in which substantially all mixing of the fermentation broth is accomplished by release of the bubbles of the gas by the spargers.

50. The fermentation method of any one of claims 40-49, wherein the spargers comprise three or more spargers.

51. The fermentation method of any one of claims 40-50, wherein L is equal to or greater than 2D.

52. The fermentation method of claim 51, wherein the spargers comprise a number of spargers equal to L/D rounded up or down to an integer number.

53. The fermentation method of claim 52, wherein the spargers are spaced apart from one another along the straight wall length L of the fermentation vessel by a distance within 20% of D.

54. The fermentation method of claim 52, wherein the spargers are spaced apart from one another along the straight wall length L of the fermentation vessel by a distance within 10% of D.

55. The fermentation method of claim 52, wherein the spargers are spaced apart from one another along the straight wall length L of the fermentation vessel by a distance within 5% of D.

56. The fermentation method of claim 52, wherein the spargers are spaced apart from one another along the straight wall length L of the fermentation vessel by a distance of D.

57. The fermentation method of any one of claims 40-56, wherein at least one of the spargers comprises a double-ring sparger.

58. The fermentation method of any one of claims 40-57, wherein introducing the gas comprises introducing a first gas and a second gas, at least one of the first and second gases comprising the reactive gaseous component.

59. The fermentation method of claim 58, wherein at least one of the spargers introduces bubbles including a mixture of the first gas and the second gas into the fermentation broth.

60. The fermentation method of claim 58 or 59, wherein at least one of the spargers introduces bubbles including a different mixture of the first gas and the second gas than does at least one other of the spargers.

61. The fermentation method of claim any one of claims 58-60, wherein the first gas is air and the second gas is substantially pure oxygen.

62. The fermentation method of any one of claims 40-57, wherein the gas is air.

63. The fermentation method of any one of claims 40-57, wherein the gas is substantially pure oxygen.

64. The fermentation method of any one of claims 40-63, wherein the reactive gaseous component is selected from the group consisting of oxygen, methane, carbon monoxide, carbon dioxide, nitrogen, and hydrogen.

65. The fermentation method of claim 64, wherein the reactive gaseous component is oxygen.

66. The fermentation method of claim 64, wherein the reactive gaseous component is carbon dioxide.

67. The fermentation method of any one of claims 40-66, further comprising adjusting an introduction rate of the reactive gaseous component by at least one of the spargers as a function of time.

68. The fermentation method of claim 67, comprising adjusting the introduction rate of the reactive gaseous component by each of the spargers as a function of time.

69. The fermentation method of claim 68, wherein responsive to the adjustment of the introduction rate of the reactive gaseous component, a microbial organism in the fermentation broth favors a biological pathway producing a product.

70. The fermentation method of claim 69, wherein the product is selected from the group consisting of 1,4-butanediol, 1,3-butanediol, caprolactam, adipic acid, and 6-amino-caproic acid.

71. The fermentation method of any one of claims 40-70, wherein at least one of the spargers has a different introduction rate of the reactive gaseous component than does at least one other of the spargers.

72. The fermentation method of any one of claims 40-71, wherein each of the spargers comprises a ring sparger.

73. The fermentation method of any one of claims 40-71, wherein at least one of the spargers comprises a nozzle or pipe sparger.

74. The fermentation method of any one of claims 40-73, wherein responsive to release of the reactive gaseous component within the gas, a microbial organism in the fermentation broth produces a product.

75. The fermentation method of claim 74, wherein the product is selected from the group consisting of 1,4-butanediol, 1,3-butanediol, caprolactam, adipic acid, and 6-amino-caproic acid.

76. The fermentation method of any one of claims 74-75, wherein the microbial organism comprises a bacterium selected from the group consisting of Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

77. The fermentation method of any one of claims 74-75, wherein the microbial organism comprises a yeast or fungus selected from the group consisting of Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and Yarrowia lipolytica.

78. The fermentation method of any one of claims 74-75, wherein the microbial organism comprises algae or a methanotroph.
